(19) United States
(12) Patent Application Publication
(10) Pub. No.: US 2025/0052753 A1
(43) Pub. Date: Feb. 13, 2025

(54) SINGLE PARTICLE ANALYSIS METHOD FOR VIRAL NUCLEIC ACID VECTOR

(71) Applicant: NANOFCM ASIA PTE.LTD., Singapore (SG)

(72) Inventor: Shaobin ZHU, XIAMEN (CN)

(73) Assignee: NANOFCM ASIA PTE.LTD., Singapore (SG)

(21) Appl. No.: 18/910,055

(22) Filed: Oct. 9, 2024

(30) Foreign Application Priority Data
Aug. 10, 2023 (CN) 2023110043591

(51) Int. Cl. G01N 33/569; C12Q 1/06; G01N 15/0205; G01N 15/10; G01N 33/58

(52) U.S. Cl. CPC G01N 33/56983; C12Q 1/06; G01N 15/0211; G01N 33/582; G01N 2015/1026; G01N 2333/035; G01N 2333/15; G01N 2333/155; G01N 2333/165

(57) ABSTRACT

The present disclosure relates to a method for analyzing one or more parameters of a viral nucleic acid vector, and belongs to the field of biological technology. The method may optionally comprise preparing a particle size standard curve and/or a concentration standard solution for the viral nucleic acid vector to measure the particle size and the concentration thereof. The method comprises preparing a specific target recognition reagent conjugated to a fluorescence labeling reagent for the parameters of the viral nucleic acid vector, detecting a sample by a flow particle analyzer, and the like. The method enables a deeper analysis of the “viral titer” obtained by traditional approaches, yielding more accurate detection results, which is of significant importance for downstream release decisions, process control, and the evaluation of clinical safety.

### CROSS-REFERENCE TO RELATED APPLICATIONS

This application claims the benefit of priority from Chinese Provisional Application No. 2023110043591, filed on Aug. 10, 2023; which application is incorporated herein by reference in its entirety.

### TECHNICAL FIELD

The present disclosure belongs to the field of biological technology, and particularly relates to a method for the analysis of single viral nucleic acid vectors.

### BACKGROUND

The viral nucleic acid vector is a type of viral vector that can carry an exogenous gene after its own elements are modified by genetic engineering. Four of the most representative viral nucleic acid vectors include lentivirus vectors, retrovirus vectors, herpes simplex virus vectors, and recombinant coronavirus vectors. These vectors have the advantages of carrying large fragments of exogenous target genes, stably and continuously expressing target nucleic acids, eliciting minimal immune responses, and the like, thereby becoming the most widely used delivery vehicles for gene therapy to date, and such products have been proved to achieve clear clinical efficacy. For example, chimeric antigen receptor T-cell (CAR-T) therapy has shown a superior response rate in targeted tumor immunotherapy, and thus the FDA has approved CAR-T products based on lentivirus and retrovirus vectors (Novartis: Kymriah, Kite Pharma: Yescarta) for marketing. The herpes simplex virus vector serves as an important oncolytic virus product. In China, the first oncolytic product-orienX010 (herpes simplex virus-1, HSV-1) was approved for clinical trials in 2009, and another oncolytic product-rHSV2hGM-CSF (herpes simplex virus-2, HSV-2) was also approved for clinical trials in 2018. Recently, intensive clinical trials of non-replicating recombinant coronavirus vaccines (CN111218459A and WO2021184560) have had significant implications for the prevention and control of coronavirus-induced epidemics both currently and in the future.

In the prior art, for example, measuring lentivirus titers typically involves methods such as TCID₅₀-GFP fluorescence microscopy, enzyme-linked immunosorbent assay (ELISA), and quantitative PCR assay. The TCID₅₀-GFP fluorescence microscopy requires labeling the lentivirus vector with a green fluorescent protein gene, and after transfection of cells with the lentivirus vector, the supernatant needs to be collected, serially diluted, and observed under a fluorescence microscope to quantify the viral titer. This process is relatively cumbersome, and due to the complexity of genetic expression systems, the lentivirus titer evaluated by this method is often underestimated. ELISA evaluates the packaging efficiency by detecting the content of HIV-1 p24 protein. It is more accurate and sensitive compared with the previous method. However, this method only measures the number of lentiviral particles and does not determine the actual infectivity of the lentivirus to target cells. This method still relies on empirical numerical judgment for the calculation and conversion of the actual titer of the lentivirus, and cannot account for the individual differences in real products. Moreover, since the viral suspension inevitably contains some free p24 protein, ELISA is considered to overestimate the actual viral titer. The absolute quantitative PCR assay can determine the copy number of viral genomes per cell genome in virus-infected target cells, and can accurately reflect the titer of the lentivirus, particularly lentiviruses without marker genes, to a certain extent. However, it cannot detect the titer of other non-nucleic acid bio-functional parameters of the lentivirus. Moreover, this method is highly sensitive to contamination, and the false-positive results caused by contamination pose a significant risk. The above methods are commonly used to evaluate other viral nucleic acid vectors as well.

During the preparation of viral nucleic acid vectors, the viral titer is usually used to assess the quality of the virus. At present, the above exemplary methods only measure the titer of the lentivirus in a limited way, and the resulting detection data cannot comprehensively and truly reflect the actual titer differences caused by the bio-functional differences of different components in the lentivirus. Accurately determining the quality parameters of the viral nucleic acid vectors, with the viral titer as a central focus, becomes a bottleneck of the technical development in the field of gene therapy. There is still a need to develop a method for determining the quality index of the lentivirus, which has the advantages of high efficiency, accuracy, ease of operation, small sample volume, and good repeatability, in the field of gene therapy biotechnology.

### SUMMARY

One objective of the present disclosure is to provide a detection method for a bio-functional titer of a viral nucleic acid, achieving the simultaneous detection of multiple parameters of a single particle of a viral nucleic acid vector and the simultaneous generation of multiple technical and/or product quality parameters. The detection method can assist in the optimization of the research and development processes of the viral nucleic acid vector and the development of quality control in gene therapy biotechnology.

In a first aspect, the present disclosure provides a method for analyzing one or more parameters of a viral nucleic acid vector.

The method for analyzing one or more parameters of a viral nucleic acid vector comprises:
step (1): preparing a fluorescence labeling reagent that can specifically bind to the viral nucleic acid vector and mixing the fluorescence labeling reagent with a sample comprising the viral nucleic acid vector; the fluorescence labeling reagent comprising a nucleic acid probe or a nucleic acid stain, and at least one from a lipid membrane dye or a fluorophore-conjugated target recognition reagent, and the parameter comprising a viral capsid protein marker signal and/or a viral envelope marker signal, as well as a nucleic acid signal or a target nucleic acid signal; preferably, the parameter comprising a nucleic acid signal or a target nucleic acid signal and an envelope protein marker signal;
step (2): detecting the sample solution comprising the viral nucleic acid vector by a flow particle analyzer, and recording data in a scattering channel and/or fluorescence channel; and
step (3): analyzing the data and calculating results of the parameters,
wherein the viral nucleic acid vector comprises a lentivirus vector, a retrovirus vector, a herpes simplex virus vector, and a recombinant coronavirus vector.

In some embodiments, the parameter comprises a bio-functional titer of the viral nucleic acid vector, the bio-functional titer of the viral nucleic acid vector comprising: a titer of a viral nucleic acid vector comprising a nucleic acid, a viral capsid, and an envelope, a titer of a viral nucleic acid vector comprising a nucleic acid and an envelope, a titer of a viral nucleic acid vector comprising a nucleic acid and a viral capsid, a titer of a viral nucleic acid vector comprising a target nucleic acid, a viral capsid, and an envelope, a titer of a viral nucleic acid vector comprising a target nucleic acid and an envelope, a titer of a viral nucleic acid vector comprising a target nucleic acid and a viral capsid, and a titer of an empty capsid viral nucleic acid vector.

In some embodiments, the viral nucleic acid vector comprises a lentivirus vector, a retrovirus vector, a herpes simplex virus vector, or a recombinant coronavirus vector.

In some embodiments, the parameter comprises a bio-functional titer of the lentivirus vector, the bio-functional titer of the lentivirus vector comprising: a titer of a lentivirus vector comprising a nucleic acid, a lentivirus capsid, and an envelope, a titer of a lentivirus vector comprising a target nucleic acid, a lentivirus capsid, and an envelope, a titer of a lentivirus vector comprising a nucleic acid and a VSV-G protein, a titer of a lentivirus vector comprising a target nucleic acid and a VSV-G protein, a titer of a lentivirus vector comprising a nucleic acid and an envelope, a titer of a lentivirus vector comprising a target nucleic acid and an envelope, a titer of an empty capsid lentivirus vector, a titer of a lentivirus vector comprising a nucleic acid but lacking a VSV-G protein, a titer of a lentivirus vector comprising a target nucleic acid but lacking a VSV-G protein, a titer of a lentivirus vector comprising a nucleic acid but lacking an envelope, a titer of a lentivirus vector comprising a target nucleic acid but lacking an envelope, a titer of a lentivirus vector comprising a nucleic acid and a lentivirus capsid, a titer of a lentivirus vector comprising a target nucleic acid and a lentivirus capsid, a titer of a lentivirus vector comprising a nucleic acid but lacking a lentivirus capsid, a titer of a lentivirus vector comprising a target nucleic acid but lacking a lentivirus capsid, a titer of a lentivirus vector comprising a nucleic acid and a p24 protein, a titer of a lentivirus vector comprising a target nucleic acid and a p24 protein, a titer of a lentivirus vector comprising a nucleic acid but lacking a p24 protein, a titer of a lentivirus vector comprising a target nucleic acid but lacking a p24 protein, a titer of a lentivirus vector comprising a nucleic acid and an envelope but lacking a lentivirus capsid, a titer of a lentivirus vector comprising a target nucleic acid and an envelope but lacking a lentivirus capsid, a titer of a lentivirus vector comprising a nucleic acid and a lentivirus capsid but lacking an envelope, a titer of a lentivirus vector comprising a target nucleic acid and a lentivirus capsid but lacking an envelope, a titer of a lentivirus vector comprising a nucleic acid but lacking a lentivirus capsid and an envelope, or a titer of a lentivirus vector comprising a target nucleic acid but lacking a lentivirus capsid and an envelope.

In some embodiments, the parameter comprises a bio-functional titer of the retrovirus vector, the bio-functional titer of the retrovirus vector comprising: a titer of a retrovirus vector comprising a nucleic acid, a gag capsid protein, and an env envelope protein, a titer of a retrovirus vector comprising a target nucleic acid, a gag capsid protein, and an env envelope protein, a titer of a retrovirus vector comprising a nucleic acid and an env envelope protein, a titer of a retrovirus vector comprising a target nucleic acid and an env envelope protein, a titer of an empty capsid retrovirus vector, a titer of a retrovirus vector comprising a nucleic acid but lacking an env envelope protein, a titer of a retrovirus vector comprising a target nucleic acid but lacking an env envelope protein, a titer of a retrovirus vector comprising a nucleic acid and a gag capsid protein, a titer of a retrovirus vector comprising a target nucleic acid and a gag capsid protein, a titer of a retrovirus vector comprising a nucleic acid but lacking a gag capsid protein, and a titer of a retrovirus vector comprising a target nucleic acid but lacking a gag capsid protein.

In some embodiments, the parameter comprises a bio-functional titer of the herpes simplex virus vector, the bio-functional titer of the herpes simplex virus vector comprising: a titer of a herpes simplex virus vector comprising a nucleic acid and g envelope glycoprotein, a titer of a herpes simplex virus vector comprising a target nucleic acid and g envelope glycoprotein, a titer of an empty capsid herpes simplex virus vector, a titer of a herpes simplex virus vector comprising a nucleic acid but lacking g envelope glycoprotein, and a titer of a herpes simplex virus vector comprising a target nucleic acid but lacking g envelope glycoprotein.

In some embodiments, the g envelope glycoprotein may be any one of envelope glycoprotein gB, envelope glycoprotein gC, envelope glycoprotein gD, envelope glycoprotein gE, envelope glycoprotein gG, envelope glycoprotein gH, envelope glycoprotein gI, envelope glycoprotein gJ, envelope glycoprotein gL, envelope glycoprotein gM, and envelope glycoprotein gN, or a combination thereof.

In some embodiments, the parameter comprises a bio-functional titer of the recombinant coronavirus vector, the bio-functional titer of the recombinant coronavirus vector comprising: a titer of a recombinant coronavirus vector comprising a nucleic acid and a SPIKE protein, a titer of a recombinant coronavirus vector comprising a target nucleic acid and a SPIKE protein, a titer of an empty capsid recombinant coronavirus vector, a titer of a recombinant coronavirus vector comprising a nucleic acid but lacking a SPIKE protein, and a titer of a recombinant coronavirus vector comprising a target nucleic acid but lacking a SPIKE protein.

In some embodiments, the method further comprises the following steps:
step (a): before step (2), preparing a concentration standard solution, detecting the concentration standard solution by the flow particle analyzer, and recording scattering and/or fluorescent data of the concentration standard solution and/or a sample flow rate of the flow particle analyzer to measuring the particle concentration of the sample comprising the viral nucleic acid vector; and/or
step (b): before step (2), preparing a particle size standard solution comprising particle size standards with different particle sizes, detecting the particle size standard solutions by the flow particle analyzer, and recording scattering data of the particle size standards to measure the particle size of each particle and the particle size distribution of the viral nucleic acid vector.

In some embodiments, the operation of mixing the fluorescence labeling reagent with the sample comprising the viral nucleic acid vector in step (1) may further comprise adding a surfactant to mix with the sample comprising the viral nucleic acid vector.

In some embodiments, the operation of mixing the fluorescence labeling reagent with the sample comprising the viral nucleic acid vector in step (1) may further comprise adding a surfactant to mix with the sample comprising the viral nucleic acid vector; and an antibody specifically binding to a lentivirus capsid protein is selected from a p24 antibody.

In some embodiments, the surfactant may comprise at least one of an anionic surfactant, a nonionic surfactant, an amphoteric surfactant, and a cationic surfactant. In some embodiments, the surfactant comprises at least one from a Tween surfactant, Triton-X100, SDS (sodium dodecyl sulfate), NP40 (nonylphenol polyoxyethylene ether), and a polyoxyethylene nonionic surfactant.

In some embodiments, step (a) may comprise: preparing the concentration standard solution by using beads of a known concentration.

In some embodiments, step (b) may comprise: preparing the particle size standards by using beads with the same or similar refractive index to that of virus particles, calculating a median value of each particle size standard from images acquired by an electron microscope as an accurate value of each particle size standard, and preparing the particle size standard solutions comprising the particle size standards with different particle sizes.

In some embodiments, the target recognition reagent may comprise at least one from an antibody, an antibody fragment, an antibody analog, a lectin, an aptamer, a peptide, a growth factor, a glycolipid, a polysaccharide, a ligand, and a receptor.

In some embodiments, the target recognition reagent may be an antibody, an antibody fragment or an antibody analog, a ligand, or a receptor.

In some embodiments, the antibody may comprise at least one selected from a VSV-G antibody, a gB antibody, a gC antibody, a gD antibody, a gE antibody, a gG antibody, a gH antibody, a gI antibody, a gJ antibody, a gL antibody, a gM antibody, a gN antibody, an env antibody, a SPIKE antibody, an antibody specifically binding to a lentivirus capsid protein, an antibody specifically binding to a retrovirus capsid protein, an antibody specifically binding to a herpes simplex virus capsid protein, and an antibody specifically binding to a recombinant coronavirus capsid protein.

In some embodiments, the antibody specifically binding to the lentivirus capsid protein may be selected from a p24 antibody.

In some embodiments, the antibody specifically binding to the retrovirus capsid protein may be selected from a gag antibody.

In some embodiments, the nucleic acid probe may comprise complementary sequences to a target nucleic acid or bases of the target nucleic acid, and the fluorescent dye is modified at the 5′ end or the 3′ end of the sequence or in the sequence.

In some embodiments, the nucleic acid stain comprises a cyanine dye, an impermeable dye, a permeable dye, an embedded dye, and a DNA double helix minor groove binding dye.

In some embodiments, the lipid membrane dye may be selected from a lipophilic fluorescent dye with an affinity for a cell membrane and other lipid-soluble membrane structures.

In some embodiments, the fluorescent dye is a fluorescent molecule, a fluorescent material, or a combination thereof. The fluorescent dye comprises at least one selected from an organic fluorescent molecule, a fluorescent protein, a nucleic acid dye, a lipid membrane dye, and a quantum dot.

In some embodiments, the lipid membrane dye is selected from DiD dye, DiO dye, Dil dye, DiR dye, DiA dye, Di-8-Anepps dye, Di-4-ANEPPS dye, PHK26 dye, PKH67 dye, CellMask Green dye, CellMask Orange dye, CellMask Red dye, CellVue Lavender dye, CellVue Plum dye, CellVue NIR780 dye, and carboxyfluorescein diacetate succinimdyl ester.

In some embodiments, the nucleic acid stain or the nucleic acid dye is selected from Acridine Orange, Acridinc Orange, Actinomycin D, 7-AAD (7-aminoactinomycin D), ACMA (9-amino-6-chloro-2-methoxyacridine), BOBO-1 Iodide (462/481), BOBO-3 Iodide (570/602), 1 mM solution in DMSO, DAPI (4′,6-diamidino-2-phenylindole, dihydrochloride), FluoroPure grade, dihydroethdium (hydroethidine), dihydroethdium (hydroethidine), dihydroethdium (hydroethidine), Ethidium Homodimer-1 (EthD-1), Ethidium Homodimer-2 (EthD-2), Ethidium Monoazide Bromide (EMA), Hexidium Iodide, Hoechst 33258, Pentahydrate (bis-Benzimide), Hoechst 33258, Pentahydrate (bis-Benzimide), Hoechst 33258, Pentahydrate (bis-Benzimide) FluoroPureGrade, Hoechst 33342, Trihydrochloride, Trihydrate, Hoechst 33342, Trihydrochloride, Trihydrate, Hoechst 33342, Trihydrochloride, Trihydrate-FluoroPure Grade, Hoechst 34580, LDS 751, NeuroTrace 435/455 Blue Fluorescent Nissl Stain, NeuroTrace 500/525 Green Fluorescent Nissl Stain, NeuroTrace 530/615 Red Fluorescent Nissl Stain, Neuro Trace 640/660 Deep-Red Fluorescent Nissl Stain, POPO-1 Iodide (434/456), POPO-3 Iodide (534/570), PO-PRO-1 Iodide (435/455), propidium iodide, Propidium Iodide-FluoroPure Grade, Propidium Iodide, Quant-iT OliGreen ssDNA Assay Kit, Quant-iT OliGreen ssDNA Reagent, Quant-iT PicoGreen dsDNA Assay Kit, Quant-iT PicoGreen dsDNA Assay Kit, Quant-iT PicoGreen dsDNA Reagent, Quant-iT PicoGreen dsDNA Reagent, Quant-iT RiboGreen RNA Assay Kit, Quant-iT RiboGreen RNA Reagent, RediPlate 96 RiboGreen RNA Quantitation Kit, SYBR Gold Nucleic Acid Gel Stain, SYBR Green I Nucleic Acid Gel Stain, SYBR Green I, SYBR Green II RNA Gel Stain, SYBR Safe DNA Gel Stain, SYTO 40 Blue Fluorescent Nucleic Acid Stain, SYTO 41 Blue Fluorescent Nucleic Acid Stain, SYTO Blue Fluorescent Nucleic Acid Stain Sampler Kit (SYTO dyes 40, 41, 42, 45), SYTO 9 Green Fluorescent Nucleic Acid Stain, SYTO 11 Green Fluorescent Nucleic Acid Stain, SYTO 12 Green Fluorescent Nucleic Acid Stain, SYTO 13 Green Fluorescent Nucleic Acid Stain, SYTO 14 Green Fluorescent Nucleic Acid Stain, SYTO 16 Green Fluorescent Nucleic Acid Stain, SYTO 21 Green Fluorescent Nucleic Acid Stain, SYTO 24 Green Fluorescent Nucleic Acid Stain, SYTO BC Green Fluorescent Nucleic Acid Stain, SYTO Green Fluorescent Nucleic Acid Stain Sampler Kit #1-SYTO dyes 11-16, SYTO 82 Orange Fluorescent Nucleic Acid Stain, SYTO 83 Orange Fluorescent Nucleic Acid Stain, SYTO 84 Orange Fluorescent Nucleic Acid Stain, SYTO 85 Orange Fluorescent Nucleic Acid Stain, SYTO Orange Fluorescent Nucleic Acid Stain Sampler Kit-SYTO dyes, SYTO 17 Red Fluorescent Nucleic Acid Stain, SYTO 59 Red Fluorescent Nucleic Acid Stain, SYTO 60 Red Fluorescent Nucleic Acid Stain, SYTO 61 Red Fluorescent Nucleic Acid Stain, SYTO 62 Red Fluorescent Nucleic Acid Stain, SYTO 63 Red Fluorescent Nucleic Acid Stain, SYTO 64 Red Fluorescent Nucleic Acid Stain, SYTO Red Fluorescent Nucleic Acid Stain Sampler Kit-SYTO dyes 17 and 59-64, SYTO RNASelect Green Fluorescent cell Stain, SYTOX Blue Dead Cell Stain, SYTOX Blue Nucleic Acid Stain, SYTOX Green Dead Cell Stain, SYTOX Green Nucleic Acid Stain, SYTOX Orange Nucleic Acid Stain, SYTOX Orange Dead Cell Stain, SYTOX Red Dead Cell Stain, SYTOX Dead Cell Stain Sampler Kit, TO-PRO-1 Iodide (515/531), TO-PRO-3 Iodide (642/661), TOTO-1 Iodide (514/533), TOTO-3 Iodide (642/660), YO-PRO-1 Iodide (491/509), YO-PRO-3 Iodide (612/631), YOYO-1 Iodide (491/509), YOYO-3 Iodide (612/631), HCS NuclearMask Deep Red Stain, HCS NuclearMask Blue Stain, HCS NuclearMask Red Stain, and UltraPure ethidium bromide.

In some embodiments, the target recognition reagent is an antibody comprising at least one of a VSV-G antibody, an env envelope protein antibody, a g envelope glycoprotein antibody, a SPIKE protein antibody, a gag antibody, a p24 antibody, a viral capsid protein antibody, or an envelope protein antibody.

In some embodiments, the target recognition reagent is a ligand and/or receptor, and the ligand and/or receptor is at least one of a VSV-G ligand and/or receptor, an env envelope protein ligand and/or receptor, a g envelope glycoprotein ligand and/or receptor, a SPIKE protein ligand and/or receptor, a gag ligand and/or receptor, a p24 ligand and/or receptor, a viral capsid protein ligand and/or receptor, or an envelope protein ligand and/or receptor.

In some embodiments, the fluorescent dye comprises a fluorescent molecule, a fluorescent material, or a combination thereof, and the fluorescent dye is directly conjugated to the target recognition reagent; or the fluorescent dye is indirectly conjugated to the target recognition reagent with a recognition group comprising an antibody, an antigen, a receptor, or a polysaccharide, or any other particles or molecules, or any portions thereof, and the recognition group is capable of specifically recognizing the target recognition reagent.

In some embodiments, mixing the fluorescence labeling reagent with the sample comprising the viral nucleic acid vector in step (1) comprises mixing the fluorescence labeling reagent and the surfactant with the sample comprising the viral nucleic acid vector.

In some embodiments, step (2) may comprise photographing and counting the sample, and measuring a signal intensity value of each particle with a specific optical characteristic by the flow particle analyzer.

In some embodiments, step (2) may comprise using a flow particle analyzer with a scattered channel and at least two fluorescence channels or a flow particle analyzer with at least two fluorescence channels for detection, wherein one fluorescence channel is used for characterizing the nucleic acid or target nucleic acid signal, and the other fluorescence channel is used for characterizing the viral envelope marker signal; analyzing fluorescence signals of particles in a population with event-positive signals; recording and gating a dot plot, wherein
particles in a population with event signals, nucleic acid signals, and viral envelope marker signals are the viral nucleic acid vectors comprising the nucleic acids and the envelopes, or particles in a population with event signals, target nucleic acid signals, and viral envelope marker signals are the viral nucleic acid vectors comprising the target nucleic acids and the envelopes;
particles in a population with event signals and viral envelope marker signals, but no nucleic acid signals are the empty capsids of the viral nucleic acid vectors;
particles in a population with event signals and nucleic acid signals, but no viral envelope marker signals are viral nucleic acid vectors comprising nucleic acids but lacking envelopes, or particles in a population with event signals and target nucleic acid signals, but no viral envelope marker signals are viral nucleic acid vectors comprising target nucleic acids but lacking envelopes.

In some embodiments, the viral nucleic acid vector is the lentivirus vector, and step (2) comprises using a flow particle analyzer with a scattered channel and at least two fluorescence channels or a flow particle analyzer with at least two fluorescence channels for detection, wherein one fluorescence channel is used for characterizing the nucleic acid or target nucleic acid signal, and the other fluorescence channel is used for characterizing the viral envelope marker signal; analyzing the fluorescence signals of particles in a population with event-positive signals; recording and gating a dot plot, wherein
particles in a population with event signals, nucleic acid signals, and VSV-G signals are the lentivirus vectors comprising the nucleic acids and the VSV-G proteins, or particles in a population with event signals, target nucleic acid signals, and VSV-G signals are the lentivirus vectors comprising the target nucleic acids and the VSV-G proteins;
particles in a population with event signals and VSV-G signals, but no nucleic acid signals are the empty capsids of the lentivirus vectors;
particles in a population with event signals and nucleic acid signals, but no VSV-G signals are the lentivirus vectors comprising the nucleic acids but lacking VSV-G proteins, or particles in a population with event signals and target nucleic acid signals, but no VSV-G signals are the lentivirus vectors comprising the target nucleic acids but lacking VSV-G proteins.

In some embodiments, the viral nucleic acid vector is the retrovirus vector, and step (2) comprises using a flow particle analyzer with a scattered channel and at least two fluorescence channels or a flow particle analyzer with at least two fluorescence channels for detection, wherein one fluorescence channel is used for characterizing the nucleic acid or target nucleic acid signal, and the other fluorescence channel is used for characterizing the viral envelope marker signal; analyzing the fluorescence signals of particles in a population with event-positive signals; recording and gating a dot plot, wherein
particles in a population with event signals, nucleic acid signals, and env envelope protein signals are the retrovirus vectors comprising the nucleic acids and the env envelope proteins, or particles in a population with event signals, target nucleic acid signals, and env envelope protein signals are the retrovirus vectors comprising the target nucleic acids and the env envelope proteins;
particles in a population with event signals and env envelope protein signals, but no nucleic acid signals are the empty capsids of the retrovirus vectors;
particles in a population with event signals and nucleic acid signals, but no env envelope protein signals are the retrovirus vectors comprising the nucleic acids but lacking the env envelope proteins, or particles in a population with event signals and target nucleic acid signals, but no env envelope protein signals are the retrovirus vectors comprising the target nucleic acids but lacking the env envelope proteins.

In some embodiments, the viral nucleic acid vector is the herpes simplex virus vector, and step (2) comprises using a flow particle analyzer with a scattered channel and at least two fluorescence channels or a flow particle analyzer with at least two fluorescence channels for detection, wherein one fluorescence channel is used for characterizing the nucleic acid or target nucleic acid signal, and the other fluorescence channel is used for characterizing the viral envelope marker signal; analyzing the fluorescence signals of particles in a population with event-positive signals; recording and gating a dot plot, wherein
particles in a population with event signals, nucleic acid signals, and g envelope glycoprotein signals are the herpes simplex virus vectors comprising the nucleic acids and the g envelope glycoproteins, or particles in a population with event signals, target nucleic acid signals, and g envelope glycoprotein signal are the herpes simplex virus vectors comprising the target nucleic acids and the g envelope glycoproteins;
particles in a population with event signals and g envelope glycoprotein signals, but no nucleic acid signals are the empty capsids of the herpes simplex virus vectors;
particles in a population with event signals and nucleic acid signals, but no g envelope glycoprotein signals are the herpes simplex virus vectors comprising the nucleic acids but lacking the g envelope glycoproteins, or particles in a population with event signals and target nucleic acid signals, but no g envelope glycoprotein signal are the herpes simplex virus vectors comprising the target nucleic acids but lacking the g envelope glycoproteins.

In some embodiments, the viral nucleic acid vector is the recombinant coronavirus vector, and step (2) comprises using a flow particle analyzer with a scattered channel and at least two fluorescence channels or a flow particle analyzer with at least two fluorescence channels for detection, wherein one fluorescence channel is used for characterizing the nucleic acid or target nucleic acid signal, and the other fluorescence channel is used for characterizing the viral envelope marker signal; analyzing the fluorescence signals of particles in a population with event-positive signals; recording and gating a dot plot, wherein
particles in a population with event signals, nucleic acid signals, and SPIKE protein signals are the recombinant coronavirus vectors comprising the nucleic acids and the SPIKE proteins, or particles in a population with event signals, target nucleic acid signals, and SPIKE protein signals are the recombinant coronavirus vectors comprising the target nucleic acids and the SPIKE proteins;
particles in a population with event signals and SPIKE protein signals, but no nucleic acid signals are the empty capsids of the recombinant coronavirus vectors;
particles in a population with event signals and nucleic acid signals, but no SPIKE protein signals are the recombinant coronavirus vectors comprising the nucleic acids but lacking the SPIKE proteins, or particles in a population with event signals and target nucleic acid signals, but no SPIKE protein signals are the recombinant coronavirus vectors comprising the target nucleic acids but lacking the SPIKE proteins.

In some embodiments, step (2) may comprise using a flow particle analyzer with a scattered channel and at least two fluorescence channels or a flow particle analyzer with at least two fluorescence channels for detection, wherein one fluorescence channel is used for characterizing the nucleic acid signal or the target nucleic acid signal, and the other fluorescence channel is used for characterizing the viral capsid protein marker signal; analyzing the fluorescence signals of particles in a population with event-positive signals; recording and gating a dot plot, wherein
particles in a population with event signals, nucleic acid signals, and viral capsid protein marker signals are the viral nucleic acid vectors comprising the nucleic acids and the lentivirus capsids, or particles in a population with event signals, target nucleic acid signals, and viral capsid protein marker signals are the viral nucleic acid vectors comprising the target nucleic acids and the viral capsids;
particles in a population with event signals and viral capsid protein marker signals, but no nucleic acid signals are the empty capsids of the viral nucleic acid vectors;
particles in a population with event signals and nucleic acid signals, but no viral capsid protein marker signals are viral nucleic acid vectors comprising nucleic acids but lacking viral capsids, or particles in a population with event signals and target nucleic acid signals, but no viral capsid protein marker signal are viral nucleic acid vectors comprising target nucleic acids but lacking viral capsids.

In some embodiments, the viral nucleic acid vector is the lentivirus vector, and step (2) comprises using a flow particle analyzer with a scattered channel and at least two fluorescence channels or a flow particle analyzer with at least two fluorescence channels for detection, wherein preferably, one fluorescence channel is used for characterizing the nucleic acid signal or the target nucleic acid signal, and the other fluorescence channel is used for characterizing a lentivirus p24 protein signal when detecting the lentivirus vector; analyzing the fluorescence signals of particles in a population with event-positive signals; recording and gating a dot plot, wherein
particles in a population with event signals, nucleic acid signals, and lentivirus p24 protein signals are the lentivirus vectors comprising the nucleic acids and the lentivirus p24 proteins, or particles in a population with event signals, target nucleic acid signals, and lentivirus p24 protein signals are the lentivirus vectors comprising the target nucleic acids and the p24 proteins;
particles in a population with event signals and lentivirus p24 protein signals, but no nucleic acid signals are the empty capsids of the lentivirus vectors;
particles in a population with event signals and nucleic acid signals, but no lentivirus p24 protein signals are the lentivirus vectors comprising the nucleic acids but lacking the p24 proteins, or particles in a population with event signals and target nucleic acid signals, but no p24 protein signals are the lentivirus vectors comprising the target nucleic acids but lacking the p24 proteins.

In some embodiments, the viral nucleic acid vector is the retrovirus vector, and step (2) comprises using a flow particle analyzer with a scattered channel and at least two fluorescence channels or a flow particle analyzer with at least two fluorescence channels for detection, wherein preferably, one fluorescence channel is used for characterizing the nucleic acid signal or the target nucleic acid signal, and the other fluorescence channel is used for characterizing a gag capsid protein signal when detecting the retrovirus vector; analyzing the fluorescence signals of particles in a population with event-positive signals; recording and gating a dot plot, wherein
particles in a population with event signals, nucleic acid signals, and gag capsid protein signals are the retrovirus vectors comprising the nucleic acids and the gag capsid proteins, or particles in a population with event signals, target nucleic acid signals, and gag capsid protein signals are the retrovirus vectors comprising the target nucleic acids and the gag capsid proteins;
particles in a population with event signals and gag capsid protein signals, but no nucleic acid signals are the empty capsids of the retrovirus vectors;
particles in a population with event signals and nucleic acid signals, but no gag capsid protein signals are the retrovirus vectors comprising the nucleic acids but lacking the gag capsid proteins, or the particles in the population with event signals and target nucleic acid signals, but no gag capsid protein signals are the retrovirus vectors comprising the target nucleic acids but lacking the gag capsid proteins.

In some embodiments, step (2) may comprise using a flow particle analyzer with a scattered channel and at least one or at least two fluorescence channels or a flow particle analyzer with at least one or at least two fluorescence channels for multi-parameter detection, wherein at least one fluorescence channel is used for characterizing the nucleic acid signal or the target nucleic acid signal, and other fluorescence channels are used for characterizing at least one viral capsid protein marker signal or at least one viral envelope marker signal; analyzing the fluorescence signals of particles in a population with event-positive signals; recording detection results in single or multiple dot plots; and gating the obtained dot plots, wherein
particles in a population with event signals, nucleic acid signals, viral capsid protein marker signals, and viral envelope marker signals are the viral nucleic acid vectors comprising the nucleic acids, the viral capsids, and the envelopes, or particles in a population with event signals, target nucleic acid signals, viral capsid protein marker signals, and viral envelope marker signals are the viral nucleic acid vectors comprising the target nucleic acids, the viral capsids, and the envelopes;
particles in a population with event signals but no nucleic acid signals are the empty capsids of the viral nucleic acid vectors, the particles in the population of the empty capsids of the viral nucleic acid vectors comprising at least one of the following particle populations: a) particles in a population of the empty capsids of viral nucleic acid vector with event signals and viral envelope marker signals, but no nucleic acid signals and viral capsid protein marker signals; b) particles in a population of the empty capsids of viral nucleic acid vectors with event signals and viral capsid protein marker signals, but no nucleic acid signals and viral envelope marker signals; c) particles in a population of the empty capsids of viral nucleic acid vectors with event signals, viral capsid protein marker signals, and viral envelope marker signals, but no nucleic acid signals; and d) particles in a population of the empty capsids of viral nucleic acid vector with event signals, but no nucleic acid signals, viral capsid protein marker signals, and viral envelope marker signals;
particles in a population with event signals, nucleic acid signals, and viral envelope marker signals, but no viral capsid protein marker signals are viral nucleic acid vectors comprising nucleic acids and envelopes but lacking viral capsids; or particles in a population with event signals, target nucleic acid signals, and viral envelope marker signals, but no viral capsid protein marker signals are viral nucleic acid vectors comprising target nucleic acids and envelopes but lacking viral capsids;
particles in a population with event signals, nucleic acid signals, and viral capsid protein marker signals, but no viral envelope marker signals are viral nucleic acid vectors comprising nucleic acids and capsids but lacking envelopes, or particles in a population with event signals, target nucleic acid signals, and viral capsid protein marker signals, but no viral envelope marker signals are viral nucleic acid vectors comprising target nucleic acids and viral capsids but lacking envelopes;
particles in a population with event signals and nucleic acid signals, but no viral capsid protein marker signals and viral envelope marker signals are viral nucleic acid vectors comprising nucleic acids but lacking viral capsids and envelopes, or particles in a population with event signals and target nucleic acid signals, but no viral capsid protein marker signals and viral envelope marker signals are viral nucleic acid vectors comprising target nucleic acids but lacking viral capsids and envelopes. The fluorescence channel is an apparatus that enables a target to be detected to emit a fluorescence signal after absorbing light waves with specific wavelengths by means of emitted light, and different fluorescence channels may be obtained by different light splitting means.

In some embodiments, step (a) may comprise detecting and recording the concentration standard solution and the sample solution comprising the viral nucleic acid vector at the same sample injection pressure in the same detection time by the flow particle analyzer, and
a calculation method for the particle concentration in the viral nucleic acid vector is as follows:
the particle concentration in the viral nucleic acid vector (particles/mL)=c×A1/A2,
wherein,
c is a concentration of the concentration standard solution, particles/mL;
A1 is the number of particles with event signals in the same unit time as A2 in the sample solution comprising the viral nucleic acid vector obtained by the detection gating from a scattered channel;
A2 is the number of particles with event signals in the same unit time as A1 in the concentration standard solution obtained by the detection gating from the scattered channel.

In some embodiments, step (a) may comprise detecting and recording the concentration standard solution and the sample solution comprising the viral nucleic acid vector at the same sample injection pressure in the same detection time by the flow particle analyzer, and
the calculation method for the particle concentration in the viral nucleic acid vector is as follows:
the particle concentration in the viral nucleic acid vector (particles/mL)=A1/K,
wherein,
K is a volumetric flow rate of the flow particle analyzer in a unit time;
A1 is the number of particles with event signals in the same unit time after conversion in the sample solution comprising the viral nucleic acid vector obtained by the detection gating in the scattered channel.

In some embodiments, step (b) may comprise detecting and recording the particle size standard solutions comprising the particle size standards with different particle sizes and the sample solution comprising the viral nucleic acid vector using the flow particle analyzer with the same detection settings and the same detection time, the detection settings comprising laser power and attenuation coefficient in a scattered channel; a calculation method for the particle size and the particle size distribution of the viral nucleic acid vector is to construct a relation curve between average values of the scattered light intensity from the detection gating in the scattered channel and the particle size of the particle size standards, and then to calculate the particle size and the particle size distribution in the sample solution comprising the viral nucleic acid vector by using the scattered light intensity from the detection gating in the scattered channel of the viral nucleic acid vector in the sample solution and the constructed curve above.

In some embodiments, a calculation method for a titer of a viral nucleic acid vector comprising a nucleic acid and an envelope protein may be to multiply P1 and the particle concentration in the viral nucleic acid vector, wherein P1 is a percentage of the number of particles in a population with event signals, nucleic acid signals, and envelope protein signals in the total number of particles with event signals.

In some embodiments, a calculation method for the titer of the viral nucleic acid vector comprising the nucleic acid and the envelope may be to multiply P1a and the particle concentration in the viral nucleic acid vector, wherein P1a is a percentage of the number of the particles in the population with the event signals, the nucleic acid signals, and the viral envelope marker signals in the total number of the particles with the event signals.

In some embodiments, a calculation method for the titer of the empty capsid lentivirus vector may be to multiply P2 and the particle concentration in the viral nucleic acid vector, wherein P2 is a percentage of the number of particles in a population with event signals and envelope protein signals, but no nucleic acid signals in the total number of the particles with the event signals; or P2 is a percentage of the number of the particles in the population with the event signals and the viral capsid protein marker signals, but no nucleic acid signals in the total number of the particles with the event signals; or P2 is a percentage of the number of the particles in the population of the empty capsids of the lentivirus vectors with the event signals and the viral envelope marker signals, but no nucleic acid signals and viral capsid protein marker signals in the total number of the particles with the event signals; or P2 is a percentage of the number of the particles in the population of the empty capsids of the viral nucleic acid vectors with the event signals and the viral capsid protein marker signals, but no nucleic acid signals and viral envelope marker signals in the total number of the particles with the event signals; or P2 is a percentage of the number of the particles in the population of the empty capsids of the viral nucleic acid vectors with the event signals, the viral capsid protein marker signals, and the viral envelope marker signals, but no nucleic acid signals in the total number of the particles with the event signals; or P2 is a percentage of the number of the particles in the population of the empty capsids of the viral nucleic acid vectors with the event signals, but no nucleic acid signals, viral capsid protein marker signals, and viral envelope marker signals in the total number of the particles with the event signals.

In some embodiments, a calculation method for a titer of the viral nucleic acid vector comprising the nucleic acid but lacking the envelope may be to multiply P3 and the particle concentration in the viral nucleic acid vector, wherein P3 is a percentage of the number of particles with event signals and nucleic acid signals, but no envelope signals in the total number of the particles with the event signals.

In some embodiments, a calculation method for the titer of the viral nucleic acid vector comprising the nucleic acid but lacking the envelope may be to multiply P4 and the particle concentration in the viral nucleic acid vector, wherein P4 is a percentage of the number of the particles with the event signals and the nucleic acid signals, but no viral envelope marker signals in the total number of the particles with the event signals.

In some embodiments, a calculation method for a titer of the viral nucleic acid vector comprising the nucleic acid but lacking the viral capsid may be to multiply P5 and the particle concentration in the viral nucleic acid vector, wherein P5 is a percentage of the number of the particles with the event signals and the nucleic acid signals, but no viral capsid protein marker signals in the total number of the particles with the event signals.

In some embodiments, a calculation method for a titer of a viral nucleic acid vector comprising a nucleic acid but lacking a capsid protein may be to multiply P6 and the particle concentration in the viral nucleic acid vector, wherein P6 is a percentage of the number of particles with event signals and nucleic acid signals, but no viral capsid protein signals in the total number of the particles with the event signals.

In some embodiments, a calculation method for the titer of the lentivirus vector comprising the nucleic acid and the envelope but lacking the viral capsid may be to multiply P7 and the particle concentration in the viral vector, wherein P7 is a percentage of the number of the particles in the population with the event signals, the nucleic acid signals, and the viral envelope signals, but no viral capsid protein signals in the total number of the particles with the event signals.

In some embodiments, a calculation method for a titer of the viral nucleic acid vector comprising the nucleic acid and the viral capsid but lacking the virus envelope may be to multiply P8 and the particle concentration in the viral nucleic acid vector, wherein P8 is a percentage of the number of the particles in the population with the event signals, the nucleic acid signals, and the viral capsid protein signals, but no viral envelope signals in the total number of the particles with the event signals.

In some embodiments, a calculation method for a titer of the viral nucleic acid vector comprising the nucleic acid but lacking the viral capsid and the envelope may be to multiply P9 and the particle concentration in the viral vector, wherein P9 is a percentage of the number of the particles in the population with the event signals and the nucleic acid signals, but no viral capsid protein signals and viral envelope signals in the total number of the particles with the event signals.

In some embodiments, a calculation method for a titer of a viral nucleic acid vector comprising a target nucleic acid and an envelope protein may be to multiply P1b and the particle concentration in the viral nucleic acid vector, wherein P1b is a percentage of the number of the particles in the population with the event signals, the target nucleic acid signals, and the envelope protein signals in the total number of the particles with the event signals.

In some embodiments, a calculation method for the titer of the viral nucleic acid vector comprising the target nucleic acid and the envelope may be to multiply Plc and the particle concentration in the viral nucleic acid vector, wherein Plc is a percentage of the number of the particles in the population with the event signals, the target nucleic acid signals, and the viral envelope marker signals in the total number of the particles with the event signals.

In some embodiments, a calculation method for a titer of a viral nucleic acid vector comprising a target nucleic acid but lacking an envelope protein may be to multiply P3a and the particle concentration in the viral nucleic acid vector, wherein P3a is a percentage of the number of particles with event signals and target nucleic acid signals, but no envelope protein signals in the total number of the particles with the event signals.

In some embodiments, a calculation method for the titer of the viral nucleic acid vector comprising the target nucleic acid but lacking the envelope may be to multiply P4a and the particle concentration in the viral nucleic acid vector, wherein P4a is a percentage of the number of the particles with the event signals and the target nucleic acid signals, but no viral envelope marker signals in the total number of the particles with the event signals.

In some embodiments, a calculation method for a titer of the viral nucleic acid vector comprising the target nucleic acid but lacking the viral capsid may be to multiply P5a and the particle concentration in the viral nucleic acid vector, wherein P5a is a percentage of the number of the particles with the event signals and the target nucleic acid signals, but no viral capsid protein marker signals in the total number of the particles with the event signals.

In some embodiments, a calculation method for a titer of a viral nucleic acid vector comprising a target nucleic acid but lacking a viral capsid protein is to multiply P6a and the particle concentration in the viral nucleic acid vector, wherein P6a is a percentage of the number of particles with event signals and target nucleic acid signals, but no viral capsid protein signals in the total number of the particles with the event signals.

In some embodiments, a calculation method for a titer of a viral nucleic acid vector comprising a target nucleic acid and an envelope but lacking a viral capsid protein may be to multiply P7a and the particle concentration in the viral nucleic acid vector, wherein Pa is a percentage of the number of particles in a population with event signals, target nucleic acid signals, and viral envelope signals, but no viral capsid protein signals in the total number of the particles with the event signals.

In some embodiments, a calculation method for a titer of the viral nucleic acid vector comprising the target nucleic acid and the viral capsid but lacking the viral envelope may be to multiply P8a and the particle concentration in the viral nucleic acid vector, wherein P8a is a percentage of the number of the particles in the population with the event signals, the target nucleic acid signals, and the viral capsid protein signals, but no viral envelope signals in the total number of the particles with the event signals.

In some embodiments, a calculation method for a titer of the viral nucleic acid vector comprising the target nucleic acid but lacking the viral capsid and the envelope may be to multiply P9a and the particle concentration in the viral nucleic acid vector, wherein P9a is a percentage of the number of the particles in the population with the event signals and the target nucleic acid signals, but no viral capsid protein signals and viral envelope signals in the total number of the particles with the event signals.

In some embodiments, the flow particle analyzer is a particle analysis detection instrument capable of achieving directional flow of a sample flow.

In some embodiments, a directional fluid system consists of a sample loading unit and a flow unit.

In some embodiments, the particle analysis detection instrument comprises an optical system and a particle detector; the optical system is used for providing a light source for irradiating sample particle populations, and collecting light rays emitted by the light source or transmitting or blocking the light rays based on light ray-associated emission angles.

In some embodiments, the particle detector consists of a photoelectric sensor and a signal conditioning circuit with a function of band-limited filtering of high-frequency noise; the signal conditioning circuit with the function of band-limited filtering of high-frequency noise is used for acquiring information of the particle populations after being irradiated by the light source.

In some embodiments, the nucleic acid signal, the target nucleic acid signal, the viral envelope protein marker signal, the lentivirus vector VSV-G signal, the retrovirus vector envelope protein signal, the herpes simplex virus vector g envelope glycoprotein signal, or the recombinant coronavirus SPIKE protein signal represents a particle signal located on the right side or the upper side of a gating line in the gate arranged in the obtained result dot plot for the corresponding index. No nucleic acid signal, no target nucleic acid signal, no lentivirus envelope protein marker signal, no lentivirus vector VSV-G signal, no retrovirus vector envelope protein signal, no herpes simplex virus vector g envelope glycoprotein signal, or no recombinant coronavirus SPIKE protein signal represents a particle signal located on the left side or the lower side of the gating line in the gate arranged in the obtained result dot plot for the corresponding index.

In some embodiments, the scattered light signal refers to a particle signal value of a scattered light signal generated by particles having a particle size of not less than 30 nm and not more than 600 nm in the process of determining the concentration-particle size distribution of the viral nucleic acid vector sample in the flow cytometry particle population.

In some embodiments, the scattered light-negative signal refers to a particle signal value of a scattered light signal generated by particles having a particle size of less than 30 nm in the process of determining the concentration-particle size distribution of the viral nucleic acid vector sample in the flow cytometry particle population.

In some embodiments, the event signal refers to an output signal in the form of an event, including a scattered light signal or replacing a scattered light signal with a fluorescence signal by a fluorescent labeling technique, to represent particles having a particle size of not less than 30 nm and not more than 600 nm.

In some embodiments, the event-negative signal refers to an output signal in the form of an event, including a scattered light signal or replacing a scattered light signal with a fluorescence signal by a fluorescent labeling technique, to represent particles having a particle size of less than 30 nm.

In some embodiments, the viral nucleic acid vector comprising the nucleic acid and the envelope protein, or the viral nucleic acid vector comprising the nucleic acid and the envelope, or the viral nucleic acid vector comprising the nucleic acid, the viral capsid, and the envelope is a viral nucleic acid vector with a complete packaging structure.

In some embodiments, the detection parameters for the detection in step (2) are as follows: a laser detector 488 nm+638 nm; single-laser channel detection laser: 20/50 mW, 488 nm; scattered light attenuation: 0.2%; detection pressure: 1 kpa; and signal type: large signal, and the scattered channel is used. The detection parameters are used for detecting the concentration of the retrovirus vector or lentivirus vector in the sample solution.

In some embodiments, the detection parameters for the detection in step (2) are as follows: a laser detector 488 nm+638 nm; single-laser channel detection laser: 10/50 mW, 488; scattered light attenuation: 10%; detection pressure: 1 kpa; and signal type: under the condition of a small signal, and the scattered channel and the FITC and PC5 dual fluorescence channel are used. The detection parameters are used for detecting particles of the lentivirus vector comprising the nucleic acid and the VSV-G protein or particles of the retrovirus comprising the nucleic acid and the VSV-G protein, particles of the empty capsid lentivirus vector or particles of the empty capsid retrovirus, particles of the lentivirus vector comprising the nucleic acid but lacking the VSV-G protein or particles of the retrovirus comprising the nucleic acid but lacking the VSV-G protein, and/or particles of the lentivirus vector comprising the target nucleic acid but lacking the VSV-G protein or particles of the retrovirus comprising the target nucleic acid but lacking the VSV-G protein in the sample solution.

In some embodiments, the detection parameters for the detection in step (2) are as follows: a laser detector 488 nm+638 nm; single-laser channel detection laser: 10/50 mW, 488; scattered light attenuation: 10%; detection pressure: 1 kpa; and signal type: under the condition of a small signal, the scattered channel is used, and the sampling pressure is fixed at 1.0 kPa. The detection parameters are used for detecting the particle size distribution of the retrovirus vector or lentivirus vector in the sample solution.

In some embodiments, the detection parameters for the detection in step (2) are as follows: a laser detector 488 nm+638 nm; single-laser channel detection laser: 10/50 mW, 488; scattered light attenuation: 10%; detection pressure: 1 kpa; and signal type: under the condition of a small signal, and the FITC and PC5 dual fluorescence channel is used. The detection parameters are used for detecting particles of the lentivirus vector comprising the capsid and the VSV-G protein or particles of the retrovirus comprising the capsid and the VSV-G protein, particles of the lentivirus vector comprising VSV-G protein but lacking the capsid or particles of the retrovirus comprising VSV-G protein but lacking the capsid, and/or particles of the lentivirus vector comprising the capsid but lacking the VSV-G protein or particles of the retrovirus comprising the capsid but lacking the VSV-G protein in the sample solution.

In some embodiments, the detection parameters for the detection in step (2) are as follows: a laser detector 488 nm+638 nm; single-laser channel detection laser: 10/50 mW, 488; scattered light attenuation: 10%; detection pressure: 1 kpa; and signal type: under the condition of a small signal, and the scattered channel and the PC5 fluorescence channel are used. The detection parameters are used for detecting particles of the lentivirus vector comprising the envelope or particles of the retrovirus comprising the envelope, and/or particles of the lentivirus vector lacking the envelope or particles of the retrovirus lacking the envelope in the sample solution.

In some embodiments, the detection parameters for the detection in step (2) are as follows: a laser detector 488 nm+638 nm; single-laser channel detection laser: 10/50 mW, 488; scattered light attenuation: 10%; detection pressure: 1 kpa; and signal type: under the condition of a small signal, and the FITC and PC5 dual fluorescence channel is used. The detection parameters are used for detecting particles of the herpes simplex virus vector comprising the nucleic acid and the envelope, particles of the empty capsid herpes simplex virus vector, and/or particles of the herpes simplex virus vector comprising the nucleic acid but lacking the envelope in the sample solution.

In some embodiments, the detection parameters for the detection in step (2) are as follows: a laser detector 488 nm+638 nm; single-laser channel detection laser: 10/50 mW, 488; scattered light attenuation: 10%; detection pressure: 1 kpa; and signal type: under the condition of a small signal, and the scattered channel and the FITC and PC5 dual fluorescence channel are used. The detection parameters are used for detecting particles of the herpes simplex virus vector comprising the nucleic acid and the envelope, particles of the empty capsid herpes simplex virus vector, and/or particles of the herpes simplex virus vector comprising the nucleic acid but lacking the envelope in the sample solution.

### Beneficial Effects

Compared with the prior art, a certain embodiment of the present disclosure has at least one of the following beneficial effects:

(1) By employing the method provided by the present disclosure to detect the bio-functional titer of the particle population of the viral nucleic acid vector, a deeper analysis of the “total viral titer” obtained by traditional approaches can be achieved; single particle analysis is performed on various sub-populations of the viral nucleic acid vector based on markers such as viral envelopes, capsid proteins, and glycoproteins, yielding more accurate detection results; moreover, the method is simple to operate and fast, and is of significant importance for downstream release decisions, process control, and the evaluation of clinical safety.

(2) Compared with the TCID₅₀-GFP fluorescence microscopy, the method provided by the present disclosure is simple to operate, fast, and capable of performing single particle analysis on various sub-populations of the lentivirus vector.

### BRIEF DESCRIPTION OF DRAWINGS

FIG. 1 shows a concentration-particle size distribution diagram of a concentrated solution of the lentivirus vector product in Example 1 in the flow cytometry particle population.

FIG. 2 shows detection results from the scattered channel and a dot plot of fluorescence signal particle populations with scattered light signals in Example 1.

FIG. 3 shows a standard curve of the HIV-1 p24 concentration versus absorbance and a detection result diagram of the lentivirus vector product in Comparative Example 1.

FIG. 4 shows a fluorescence microscopy observation diagram of a concentrated solution of the lentivirus vector product in Comparative Example 2.

FIG. 5 shows a comparison diagram of the results obtained from Example 1, Comparative Example 1, and Comparative Example 2 in Example 2.

FIG. 6 shows a dot plot of fluorescence signal particle populations of the FITC and PC5 fluorescence channel in Example 3.

FIG. 7 shows detection results from the scattered channel and a dot plot of fluorescence signal particle populations with scattered light signals in Comparative Example 4.

FIG. 8 shows a comparison diagram of positive rates after envelope dye labeling and after treatment with different concentrations of a membrane permeabilizer in Example 4, wherein for the horizontal coordinate, 0 represents Ox membrane permeabilizer, 0.5 represents 0.5× membrane permeabilizer, 1 represents 1× membrane permeabilizer, and 2 represents 2× membrane permeabilizer.

FIG. 9 shows a dot plot of fluorescence signal particle populations of the FITC and PC5 fluorescence channel in Example 5.

FIG. 10 shows detection results from the scattered channel after labeling with antibodies and reagents from different manufacturers and comparison diagrams of dot plots of fluorescence signal particle populations with scattered light signals in Comparative Example 5, wherein P1 represents the percentage of particles of the herpes simplex virus vector with positive expression of the envelope-specific marker; P2 represents the percentage of particles of the herpes simplex virus vector with negative expression of the envelope-specific marker.

FIG. 11 shows detection results from the scattered channel and a dot plot of fluorescence signal particle populations with scattered light signals in Example 6.

FIG. 12 shows detection results from the scattered channel and a dot plot of fluorescence signal particle populations with scattered light signals in Example 7.

FIG. 13 shows a dot plot of fluorescence signal particle populations of the FITC and PC5 fluorescence channel in Example 8.

FIG. 14 shows a dot plot of fluorescence signal particle populations of the FITC and PC5 fluorescence channel in Example 9.

### DESCRIPTION OF THE TERMS

In the present disclosure, the term “room temperature” represents ambient temperature, and may be 10-40° C., or may be 20-30° C.; in some embodiments, the room temperature is 22-28° C.; in some embodiments, the room temperature is 24-26° C.; in some embodiments, the room temperature is 25° C.

In the context of the present disclosure, all numbers disclosed herein are approximate values, whether or not the word “about” or “approximately” is used. Based on the disclosed numbers, it is possible that the numerical value of each number may have a difference of +10% or less or a reasonable difference considered by those skilled in the art, such as by +1%, +2%, +3%, +4%, or +5%.

The term “more” refers to a quantity of 2 or more.

The term “titer” refers to the number of viral particles with bioactivity per milliliter.

The term “TU” refers to the abbreviation of “transducing units”, and represents the number of viral genomes that can infect and enter target cells. The term “viral nucleic acid vector comprising an envelope” refers to a type of viral nucleic acid vector with a lipid-based membrane structure, which is attached to and encapsulates the outer side of the nucleocapsid.

The term “gating” refers to setting gates, and means that a specific range or region is defined within a flow cytometry particle distribution diagram, and each particle population is analyzed based on single or multiple parameters. The shapes of gates include linear gates, quadrant gates, rectangular gates, circular gates, polygonal gates, arbitrary shape gates, four-quadrant gates, and the like.

The term “optional” or “optionally” means that the subsequently described event or case may or may not occur. For example, “optional surfactant” means that the surfactant may or may not be present.

The term “VSV-G protein” refers to a fusion capsid G glycoprotein of a lentivirus vector.

The term “g envelope glycoprotein” refers to a glycoprotein on an envelope of a herpes simplex virus vector, including at least one of envelope glycoprotein gB, envelope glycoprotein gC, envelope glycoprotein gD, envelope glycoprotein gE, envelope glycoprotein gG, envelope glycoprotein gH, envelope glycoprotein gI, envelope glycoprotein gJ, envelope glycoprotein gL, envelope glycoprotein gM, or envelope glycoprotein gN.

The term “envelope protein” refers to an envelope glycoprotein of a retrovirus vector.

The term “SPIKE protein” refers to a spike glycoprotein of a recombinant coronavirus.

The term “and/or” should be understood to mean any one of the options or a combination of any two or more of the options.

In this specification, the reference terms, such as “one embodiment”, “some embodiments”, “examples”, “a specific example”, or “some examples”, mean that a specific feature, structure, material, or characteristic described in connection with the embodiment or example is included in at least one embodiment or example of the present disclosure. In this specification, the schematic descriptions of the terms described above do not necessarily refer to the same embodiment or example. Moreover, the specific features, structures, materials, or characteristics described may be combined in any one or more embodiments or examples in an appropriate manner. Moreover, various embodiments or examples and features of various embodiments or examples described in this specification may be combined by those skilled in the art to the extent that they do not contradict each other.

### DETAILED DESCRIPTION OF THE EMBODIMENTS

In order to make the technical solutions of the present disclosure better understood by those skilled in the art, some non-limiting examples are further disclosed below to further explain the present disclosure in detail.

The reagents used in the present disclosure are either commercially available or may be prepared by the methods described herein.

Specimen source: A concentrated solution of a lentivirus vector product was provided by a certain company, a concentrated solution of a herpes simplex virus vector product was provided by a certain company, a concentrated solution of a retrovirus vector product was provided by a certain company, 293T cells were purchased from the Center for Excellence in Molecular Cell Science, CAS, and a concentrated solution of SARS-COV-2 (2019-nCOV) Spike Pseudovirus vector was purchased from Sino Biological, Inc.

Unless otherwise specified, the buffers, solutions, or reagents of the present disclosure all adopted water as a solvent, for example, 3 μM SYTO9 represents a 3 μM aqueous SYTO9 solution; a PBS buffer represents an aqueous PBS buffer solution; and 3 μM SYTO9 in PBS buffer represents a 3 μM aqueous solution of SYTO9 in PBS buffer.

Unless otherwise specified, the preparation methods for the antibodies conjugated to fluorescent dyes in the examples or comparative examples of the present disclosure included the step of conjugating the fluorescent dyes to the antibodies according to the method in the instructions of the EZ-Link™ maleimide protein labeling kit.

### Reagents and Instruments Used:

### 1) Reagents:

Unless otherwise specified, the following reagents were selected from the following manufacturers:
SYTO Green (SYTO BC Green-Fluorescent Nucleic Acid Stains), SYTO16, SYTO21, SYTO24, PicoGreen, SYTOX Green, SYTO BC, RNA Select, SYTO60, SYTO62, and SYTO63 were purchased from Invitrogen;
p24 ELISA (QuickTiter HIV Lentivirus Quantitation Kit) was purchased from Cell Biolabs, Inc.;
Polybrene, PKH67 lipid membrane dye, and DID membrane dye were purchased from Sigma-Aldrich;
293Pro series serum-free media were purchased from Shanghai BasalMedia Technologies Co., Ltd.;
a membrane permeabilizer (Cat. No.: P2320, mother liquor (20×, active ingredient: Tween 20, 5 mL), FITC Anti-HIV1 p24 antibody (Cat. No.: NHAP24-FITC-50T), SYTO9 (100μ, Cat. No.: NHATO9-488-50T), PE-Cy5-VSVG antibody (PE-Cy5 anti-human VSVG, Cat. No.: NHAVSVG-PECy5-50T), AF647-Anti-Glycoprotein B of HSV (gB) (Cat. No.: NHAGB-AF647-50T), and a fluorescent microsphere standard were purchased from NanoFCM;
Anti-HSV1+HSV2 gB antibody [10B7] and Anti-HSV1+HSV2 gD antibody were purchased from Abcam;
HIV-1 env antibody and HIV-1 gag-pol antibody were purchased from LS bio;
AF488-Anti-Spike protein [4A8], Human IgG1, Kappa antibody was purchased from Absolute Antibody;
Herpes Simplex Virus Type 1/2 gB antibody and EZ-Link™ maleimide protein labeling kit were purchased from Thermo Fisher Scientific;
PBS buffer: an aqueous solution containing 136.89 mM NaCl, 2.67 mM KCl, 8.1 mM Na₂HPO₄, and 1.76 mM KH₂PO₄;
the concentration of the herpes simplex virus vector products (HSV-1 and HSV-2) was about 1×10⁸ PFU/mL;
the concentration of the SARS-COV-2 (2019-nCOV) Spike Pseudovirus vector product was about 1×10¹⁰ virus copies/mL;
the concentration of the retrovirus vector product was about 1×10⁹ TU/mL.

### 2) Instruments:

Flow NanoAnalyzer was purchased from NanoFCM Inc.;
Centrifuge 5810R refrigerated centrifuge was purchased from Eppendorf;
Optima Max-XP Tabletop Ultracentrifuge was purchased from Beckman Coulter;
X81 Universal Control BOX IX2 UBC 2 microscope fluorescence microscope was purchased from Olympus Corp.; and
Feyond-A300 microplate reader was purchased from Hangzhou Allsheng Instruments Co., Ltd.

### Example 1: Detection by Flow NanoAnalyzer after Labeling with Nucleic Acid Dye and VSVG Antibody Conjugated to Fluorescent Dye

### 1) Concentration Detection of Lentivirus Vector Product

Flow NanoAnalyzer detection parameters were as follows: a laser detector 488 nm+638 nm; single-laser channel detection laser: 20/50 mW, 488 nm; scattered light attenuation: 0.2%; detection pressure: 1 kpa; and signal type: large signal, and the scattered channel was used.

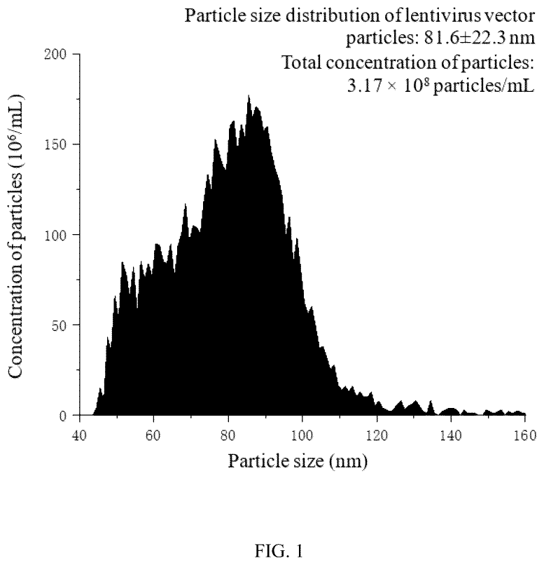

Concentration detection of the lentivirus vector product: The fluorescent microsphere standard with a concentration of 2.19×10¹⁰ particles/mL was 100-fold diluted to obtain a concentration standard solution. The concentrated solution of the lentivirus vector product was 3-fold diluted to obtain a sample solution. The concentration standard solution and the sample solution were subjected to flow cytometry particle detection with the same detection parameters, and the number of particles was recorded at the same sample injection pressure and the same detection time as the detection of the concentration standard solution. Within the detection time, the number of particles in the sample solution was 2742 and the number of particles in the concentration standard solution was 5683. The concentration of the lentivirus vector product was calculated based on the dilution factors, the concentration standard curve, and the detection results of the lentivirus vector product, resulting in the concentration of 3.17×10⁸ particles/mL (see FIG. 1).

### 2) Dual Optical Labeling of Lentivirus Vector

The concentrated solution of the lentivirus vector product was 10-fold diluted with a buffer containing PBS (containing 136.89 mM NaCl, 2.67 mM KCl, 8.1 mM Na₂HPO₄, and 1.76 mM KH₂PO₄) and 0.5% Tween 20 to obtain solution 1, and 50 μL of solution 1 was incubated with 20 μL of the PE-Cy5-VSVG antibody (a VSVG antibody conjugated to PE-Cy5) and 3 μM SYTO Green at 37° C. for 30 min. The mixture was centrifuged at 4° C. at 100000 g for 80 min, the supernatant was discarded, and the pellet was resuspended in the PBS buffer. The mixture was centrifuged at 4° C. at 100000 g for 20 min, the supernatant was discarded, and 100 μL of the PBS buffer was added to resuspend the pellet to obtain the dual-labeled lentivirus vector.

### 3) Detection of Dual-Labeled Lentivirus Vector by Flow NanoAnalyzer

Flow NanoAnalyzer detection parameters were as follows: a laser detector 488 nm+638 nm; single-laser channel detection laser: 10/50 mW, 488; scattered light attenuation: 10%; detection pressure: 1 kpa; and signal type: under the condition of a small signal, and the scattered channel and the FITC and PC5 dual fluorescence channel were used.

Detection of the dual-labeled lentivirus vector: The dual-labeled lentivirus vector was detected by the Flow NanoAnalyzer. The FITC fluorescence was a nucleic acid dye excitation signal used for representing the nucleic acid in the lentivirus vector, and the PC5 fluorescence represented the fusion capsid G glycoprotein in the lentivirus vector. The FITC fluorescence channel detection results and the PC5 fluorescence channel detection results of the particle populations with scattered light signals were analyzed, and the dot plot of the detection results was recorded. The resulting dot plot was gated into four quadrants, wherein
particles in a population with scattered light signals, nucleic acid signals, and VSV-G signals were the lentivirus vectors comprising nucleic acids and VSV-G proteins, or particles in a population with scattered light signals, target nucleic acid signals, and VSV-G signals were the lentivirus vectors comprising target nucleic acids and VSV-G proteins;
particles in a population with scattered light signals and VSV-G signals, but no nucleic acid signals were the empty capsids of the lentivirus vectors;
particles in a population with scattered light signals and nucleic acid signals, but no VSV-G signals were the lentivirus vectors comprising nucleic acids but lacking VSV-G proteins, or particles in a population with scattered light signals and target nucleic acid signals, but no VSV-G signals were the lentivirus vectors comprising target nucleic acids but lacking VSV-G proteins.

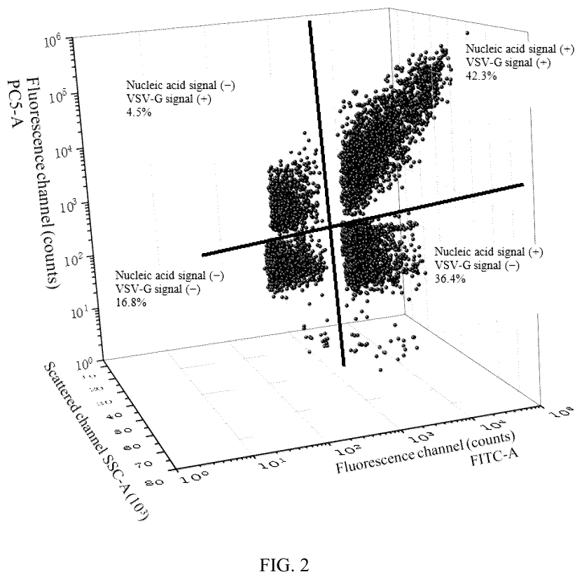

The detection results from the scattered channel and the results of the fluorescence signal particle populations with scattered light signals are shown in FIG. 2.

### 4) Calculation of Bio-Functional Titer of Lentivirus Vector Product

The particles in the population with the scattered light signals, the nucleic acid signals, and the VSV-G signals were the particles of the lentivirus vector comprising the nucleic acid and the VSV-G protein. The titer of the particles of the lentivirus vector comprising the nucleic acid and the VSV-G protein was to multiply P1 and the concentration of the lentivirus vector product obtained in step 1) of Example 1, resulting in the titer of 1.4×10⁷ TU/mL, wherein P1 is the percentage of the number of particles in the particle population with the scattered light signals, the nucleic acid signals, and the VSV-G signals in the total number of particles with scattered light signals.

The particles in the population with the scattered light signals and the VSV-G signals, but no nucleic acid signals were the particles of the empty capsids of the lentivirus vectors. The calculation method for the titer of the empty capsid lentivirus vector was to multiply P2 and the concentration of the lentivirus vector product obtained in step 1) of Example 1, resulting in the titer of 1.34×10⁸ TU/mL, wherein P2 is the percentage of the number of particles in the particle population with the scattered light signals and the VSV-G signals, but no nucleic acid signals in the total number of the particles with the scattered light signals.

The particles in the population with the scattered light signals and the nucleic acid signals, but no VSV-G signals were the particles of the lentivirus vector comprising the nucleic acid but lacking the VSV-G protein. The calculation method for the titer of the lentivirus vector comprising the nucleic acid but lacking the VSV-G protein was to multiply P3 and the concentration of the lentivirus vector product obtained in step 1), resulting in the titer of 1.15×10⁸ TU/mL, wherein P3 is the percentage of the number of the particles with the scattered light signals and the nucleic acid signals, but no VSV-G signals in the total number of the particles with the scattered light signals.

5) Detection of the particle size distribution of the lentivirus vector product: The detection parameters were set as follows: a laser detector 488 nm+638 nm; single-laser channel detection laser: 10/50 mW, 488; scattered light attenuation: 10%; detection pressure: 1 kpa; and signal type: under the condition of a small signal, and the scattered channel was used; the sampling pressure was fixed at 1.0 kPa. Under these conditions, the detection was conducted on a mixture of the particle size substances with particle sizes of 68±2 nm, 91±3 nm, 113±3 nm, and 155±3 nm, data obtained from the detection showed the median values and events values of the peaks according to the particle size standards, and a fitting curve was generated. The target particle size distribution range was defined using a gating tool for the data of the concentrated solution of the lentiviral vector product detected under the same detection parameters after 10-fold dilution. The number of particles within the gate, the percentage of the number of particles within the gate, and the median, mean, and standard deviation of the particle sizes of particles within the gate were recorded. Combined with the fitting curve, the single particle size value of the lentivirus vector product was calculated. After statistical analysis, a histogram of the particle size distribution of the lentivirus vector product was generated (FIG. 1).

### Comparative Example 1: Detection of p24 by ELISA

The procedure was performed according to the instructions of the p24 ELISA kit:

1) Gradient dilution of the HIV-1 p24 quantitative standard: A positive control containing a 320 μg/mL recombinant HIV-1 p24 antigen was subjected to serial dilution in a dilution factor with a diluent to obtain five concentrations of the positive control. The corresponding wells of the above samples were sequentially numbered, with three negative control wells, two positive control wells, and 1 blank well arranged on each plate. To each reaction well, 25 μL of lysis solution was added.

2) The concentrated solution of the lentivirus vector product was 10⁵-fold diluted to obtain sample solution 2. Sample solution 2 and the controls in step 1) of Comparative Example 1 were separately added to the reaction wells containing the lysis solution. The plate was shaken for 30-60 s for mixing well, and incubated at 37° C. for 1 h. The plate was washed 5 times and patted dry. Then an enzyme conjugate was added to each well at 125 μL/well (nothing was added to the blank well), and the plate was incubated at 37° C. for 1 h. The plate was washed 5 times and patted dry. Then a substrate (a substrate solution and a chromogenic solution were mixed at a ratio of 1:1) was added to each well at 125 μL/well. The plate was incubated at 37° C. for 30 min, and then a terminating solution was added at 50 μL/well.

3) The plate was read on a microplate reader at a wavelength of 450 nm.

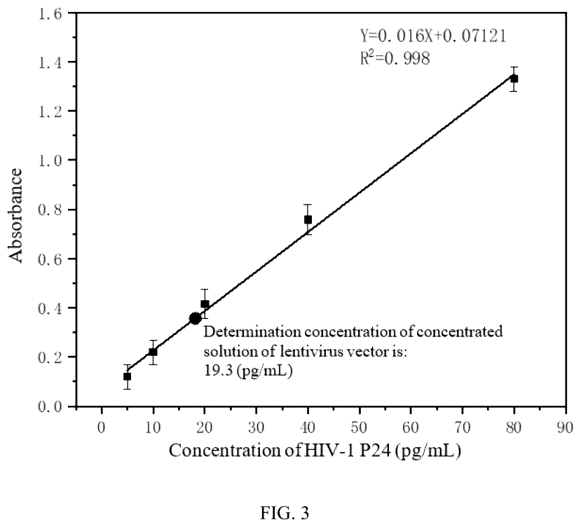

4) Positive quantification determination: Based on the five concentrations of the positive control in step 1 of Comparative Example 1, a standard curve was plotted, and the detection result of the lentivirus vector product was substituted into the curve for quantification determination. The results are shown in FIG. 3.

5) The p24 content (ng/mL) and the lentivirus vector titer (based on 1 ng p24=1.25×10⁵ TU) of the concentrated solution of the 10⁵-fold diluted lentivirus vector product were calculated according to step 4) of Comparative Example 1, resulting in the p24 content of 19.3 pg/mL of the concentrated solution of the 10⁵-fold diluted lentivirus vector product; namely, the titer of the lentivirus vector product was 19.3×10² ng/ml×1.25×10⁵ TU/mL=2.4×10⁸ TU/mL.

### Comparative Example 2: TCID₅₀-GFP Fluorescence Microscopy

1) Starting from a 103-fold dilution of the lentivirus vector product, a total of 8 dilution gradients were prepared (each differing by a factor of 10 from the previous gradient).

2) 0.05 mL of each gradient dilution in step 1 was added to a 96-well plate containing a culture medium with 293T cells (2.5×10⁶ cells/well) and 8 mg/L Polybrene (used to enhance infection) without serum. For each dilution gradient, the dilution was added to 10 wells, allowing the lentivirus to infect the 293T cells. The cells were cultured at 37° C. for 2 days.

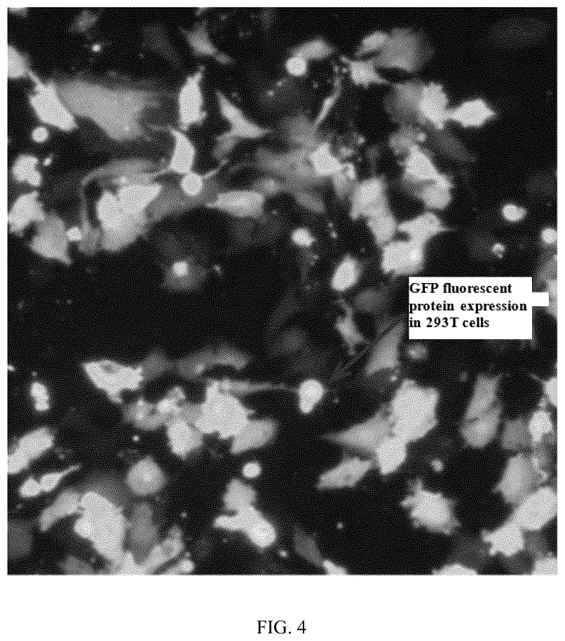

3) After 2 days, an inverted fluorescence microscope was used to observe and calculate the number of wells exhibiting green fluorescence in each row (10 wells). The results of the fluorescence microscopy for the concentrated solution of the lentivirus vector product are shown in FIG. 4.

4) Calculation of the lentivirus vector titer: It was observed that all wells in the rows corresponding to dilutions prior to the 103-fold dilution exhibited green fluorescence, while all wells in the rows corresponding to dilutions beyond the 10⁶-fold dilution exhibited no green fluorescence. At a 104-fold dilution, 3 positive wells were observed. At a 10⁵-fold dilution, 1 positive well was observed. The calculation formula was as follows:
the infectious titer T=10^{1+d (s-0.5)}/V,
where d is the logarithm of the dilution ratio (i.e., d=1 for a 10-fold dilution); s is the sum of the logarithm of the initial dilution and the positive well ratio at each test dilution, namely, 3+1+0.3+0.1=4.4; and V is the volume of the lentivirus vector product dilution for inoculation (i.e., V=0.05 mL).

Upon calculation, the infectious titer of the lentivirus vector product T=1.6×10⁶ TU/mL.

### Example 2: Comparison of Results Obtained from Example 1, Comparative Example 1, and Comparative Example 2

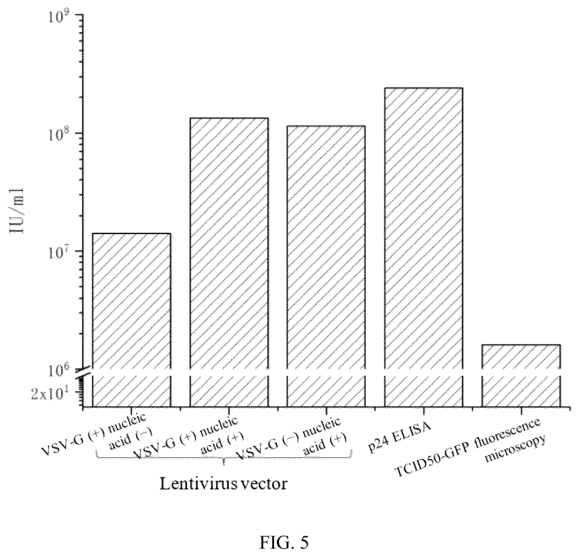

As shown in FIG. 5, it can be seen from the results that the viral titer of the concentrated solution of the lentivirus vector product detected by the p24 ELISA was often an overestimate of the actual viral titer of the sample due to the interference of free p24 and the fact that the viral particles with p24 protein expression do not represent the ability of the viral particles to specifically infect and integrate the genome into the host. In Example 1, Comparative Example 1, and Comparative Example 2, the viral titer of the concentrated solution of the lentivirus vector product detected by the p24 ELISA was 2.4×10⁸ TU/mL, which was close to the sum of the titer of the lentivirus vector comprising the nucleic acid and the VSV-G protein and the titer of the lentivirus vector comprising the nucleic acid but lacking the VSV-G protein (1.34×10⁸ TU/mL+1.15×10⁸ TU/mL=2.49×10⁸ TU/mL). Since the titer of the lentivirus vector comprising the nucleic acid but lacking the VSV-G protein was non-infectious, the data also demonstrated that the p24 ELISA overestimated the actual viral titer. The result detected by the TCID₅₀-GFP fluorescence microscopy, which was 1.6×10⁶ TU/mL, was about two orders of magnitude lower than the titer, 1.34×10⁸ TU/mL, of the lentivirus vector comprising the nucleic acid and the VSV-G protein, and the viral titer, 2.4×10⁸ TU/mL, of the concentrated solution of the lentivirus vector product detected by the p24 ELISA, suggesting that the TCID₅₀-GFP fluorescence microscopy underestimated the actual titer. Thus, the accuracy of determination of the actual viral titer of the lentivirus vector composition using the titer of the lentivirus vector comprising the nucleic acid and the VSV-G protein as the determination index was high.

### Example 3: Detection by Flow NanoAnalyzer after Dual Labeling with VSVG Antibody and P24 Antibody Conjugated to Fluorescent Dye and after Treatment with Membrane Permeabilizer

### 1) Membrane Permeabilizer Treatment and Dual Optical Labeling of Lentivirus Vector

50 μL of the lentivirus (1×10¹⁰ TU/mL) was taken, and 50 μL of the 0.5× membrane permeabilizer (the membrane permeabilizer was taken (the mother liquor (20×) was diluted with water, and other concentrations of the membrane permeabilizers below were prepared in the same manner)) was added. The mixture was mixed well, and incubated on ice for 30 min. 5 μL of the FITC-P24 antibody (manufacturer: NanoFCM) and 20 μL of the PE-Cy5-VSVG antibody (manufacturer: NanoFCM) were added. The mixture was mixed well, and incubated on ice for 60 min in the dark. The lentivirus was resuspended in 1 mL of the 0.5× membrane permeabilizer, the mixture was centrifuged at 80000 g for 30 min, and the supernatant was discarded. The lentivirus was resuspended in 1 mL of the 0.5× membrane permeabilizer again, the mixture was centrifuged at 80000 g for 30 min, and the pellet was resuspended in 100 μL of the PBS buffer to obtain the dual-labeled lentivirus vector.

### 2) Detection of Dual-Labeled Lentivirus Vector by Flow NanoAnalyzer

Flow NanoAnalyzer detection parameters were as follows: a laser detector 488 nm+638 nm; single-laser channel detection laser: 10/50 mW, 488; scattered light attenuation: 10%; detection pressure: 1 kpa; and signal type: under the condition of a small signal, and the FITC and PC5 dual fluorescence channel was used.

Detection of the dual-labeled lentivirus vector: The dual-labeled lentivirus vector was detected by the Flow NanoAnalyzer. The FITC fluorescence was a P24 fluorescent antibody excitation signal used for representing the capsid protein in the lentivirus vector, and the PC5 fluorescence represented the fusion capsid G glycoprotein in the lentivirus vector. The FITC fluorescence channel detection results and the PC5 fluorescence channel detection results were analyzed, and the dot plot of the detection results was recorded. The resulting dot plot was gated into four quadrants, wherein
particles in a population with P24 signals and VSV-G signals were lentivirus vectors comprising capsids and VSV-G proteins;
particles in a population with VSV-G protein signals and P24 protein signals were lentivirus vectors comprising VSV-G proteins but lacking capsids;
particles in a population with P24 signals but no VSV-G signals were lentivirus vectors comprising capsids but lacking VSV-G proteins.

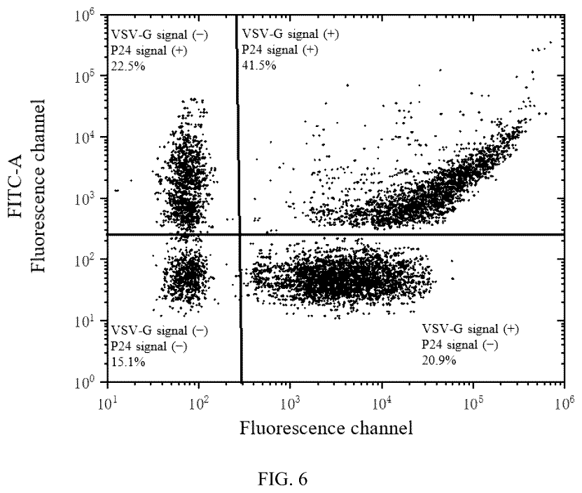

The results of the fluorescence signal particle populations are shown in FIG. 6 and Table 1.

### Comparative Example 3: Detection by Flow NanoAnalyzer after Labeling with VSVG Antibody and P24 Antibody Conjugated to Fluorescent Dye and after Treatment with Different Concentrations of Membrane Permeabilizers

According to the method provided in Example 3, the concentrations of the membrane permeabilizer were separately set to 0×, 1×, and 2×.

The comparison results of the percentage of particles of the lentivirus vector comprising the capsid and the VSV-G protein obtained by treating with different concentrations of the membrane permeabilizers are shown in Table 1.

TABLE 1
Comparison results of the percentage of particles of the lentivirus
vector comprising the capsid and the VSV-G protein obtained by treating
with different concentrations of the membrane permeabilizers

| Detection | | | | |
|---|---|---|---|---|
| method | Treatment | | | |
| (percentage | concentration | | | |
| of positive | of membrane | P24+/VSVG+ | P24−/VSVG+ | P24+/VSVG− |
| particles) | permeabilizer | (%) | (%) | (%) |
| Example 3 | 0.5x | 41.5 | 20.9 | 22.5 |
| Comparative | 0x | 14 | 40 | 10.1 |
| Example 3 | 1x | 43.8 | 21.6 | 15.5 |
| | 2x | 35.4 | 21.6 | 21.6 |

### Comparative Example 4: Detection by Flow NanoAnalyzer after Labeling with P24 Antibody Conjugated to Fluorescent Dye and after Treatment with Membrane Permeabilizer

### 1) Membrane Permeabilizer Treatment and Optical Labeling of Lentivirus Vector

50 μL of the lentivirus vector (1×10¹⁰ TU/mL) was taken, and 50 μL of the 0.5× membrane permeabilizer was added. The mixture was mixed well, and incubated on ice for 30 min. 5 μL of FITC-P24 antibody (manufacturer: NanoFCM) was added. The mixture was mixed well, and incubated on ice for 60 min in the dark. The lentivirus was resuspended in 1 mL of the 0.5× membrane permeabilizer, the mixture was centrifuged at 80000 g for 30 min, and the supernatant was discarded. The lentivirus was resuspended in 1 mL of the 0.5× membrane permeabilizer again, the mixture was centrifuged at 80000 g for 30 min, and the pellet was resuspended in 100 μL of the PBS buffer to obtain the labeled lentivirus vector.

2) Detection of dual-labeled lentivirus vector by Flow NanoAnalyzer Flow NanoAnalyzer detection parameters were as follows: a laser detector 488 nm+638 nm; single-laser channel detection laser: 10/50 mW, 488; scattered light attenuation: 10%; detection pressure: 1 kpa; and signal type: under the condition of a small signal, and the scattered channel and the FITC fluorescence channel were used.

Detection of the labeled lentivirus vector: The labeled lentivirus vector was detected by the Flow NanoAnalyzer. The FITC fluorescence was a P24 fluorescent antibody excitation signal used for representing the capsid protein in the lentivirus vector. The FITC fluorescence channel detection results were analyzed, and the dot plot of the detection results was recorded. The resulting dot plot was gated into two quadrants, wherein
particles in a population with scattered light signals and P24 signals were lentivirus vectors comprising capsids;
particles in a population with scattered light signals but no P24 signals were lentivirus vectors lacking capsids.

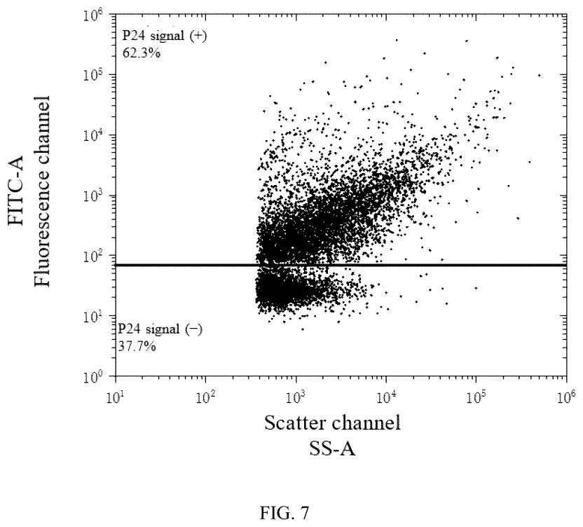

The results of the fluorescence signal particle populations with scattered light signals are shown in FIG. 7.

Conclusion: In Example 3, Comparative Example 3, and Comparative Example 4, the particles of the lentivirus vector comprising the capsid and the VSV-G protein detected in Example 3 accounted for 41.5% of the total number of particles of the lentivirus vector; whereas in Comparative Example 3, the particles of the lentivirus vector comprising the capsid and the VSV-G protein obtained by treating with the membrane permeabilizer with the concentration of 0 accounted for 14% of the total number of particles of the lentivirus vector, demonstrating that the membrane permeabilizer was critical for the penetration of fluorescent dyes into the lentivirus envelope to label the capsid protein for detection. The particles of the lentivirus vector comprising the capsid detected in Comparative Example 4 accounted for 62.3% of the total number of particles of the lentivirus vector, which was significantly higher than the percentage of the particles of the lentivirus vector simultaneously comprising the capsid and the envelope in Example 3 in the total number of particles of the lentivirus vector (41.5%). Since the lentivirus vector lacking the VSV-G protein was considered non-infectious, the data further demonstrated that the determination based solely on p24 as an index may overestimate the actual viral titer.

### Example 4: Detection by Flow NanoAnalyzer after Labeling with Envelope Dye and after Treatment with Different Concentrations of Membrane Permeabilizers

1) Envelope Dye Labeling of Lentivirus Vector Treated with Membrane Permeabilizers

1 μL of the DID membrane dye was 10-fold diluted to prepare a DID membrane dye mother liquor (prepared right before use). 50 μL of different concentrations of the membrane permeabilizers were each added and mixed well with 10 μL of the DID membrane dye mother liquor, and then the mixtures were incubated on ice for 30 min. 1 μL of the DID membrane dye mother liquor mother liquor was added to 100 μL of the lentivirus vector (with a concentration of 1×10⁸ TU/mL), and the mixture was mixed well. The mixture was incubated at 37° C. for 15 min in the dark to obtain the labeled lentivirus vector after treatment with the membrane permeabilizer. The lentivirus vector was resuspended in 1 mL of the membrane permeabilizer, the mixture was centrifuged at 80000 g for 30 min, and the supernatant was discarded. According to the above method, the concentrations of the membrane permeabilizer were set to Ox, 0.5×, 1×, and 2× to obtain labeled lentivirus vectors after treatment with different concentrations of the membrane permeabilizers (i.e., the labeled lentivirus vector after treatment with the Ox membrane permeabilizer, the labeled lentivirus vector after treatment with the 0.5× membrane permeabilizer, the labeled lentivirus vector after treatment with the 1× membrane permeabilizer, and the labeled lentivirus vector after treatment with the 2× membrane permeabilizer).

### 2) Detection of Envelope Dye-Labeled Lentivirus Vectors by Flow NanoAnalyzer

Flow NanoAnalyzer detection parameters were as follows: a laser detector 488 nm+638 nm; single-laser channel detection laser: 10/50 mW, 488; scattered light attenuation: 10%; detection pressure: 1 kpa; and signal type: under the condition of a small signal, and the scattered channel and the PC5 fluorescence channel were used.

Detection of the labeled lentivirus vectors: The labeled lentivirus vectors after treatment with different concentrations of the membrane permeabilizers obtained in step 1) of Example 4 were detected by the Flow NanoAnalyzer; the PC5 fluorescence was a DID membrane dye excitation signal used for representing the envelope in the lentivirus vector. The PC5 fluorescence channel detection results were analyzed, and the dot plot of the detection results was recorded. The resulting dot plot was gated into two quadrants, wherein
particles in a population with scattered light signals and DID membrane dye signals were lentivirus vectors comprising envelopes;
particles in a population with scattered light signals but no DID membrane dye signals were lentivirus vectors lacking envelopes.

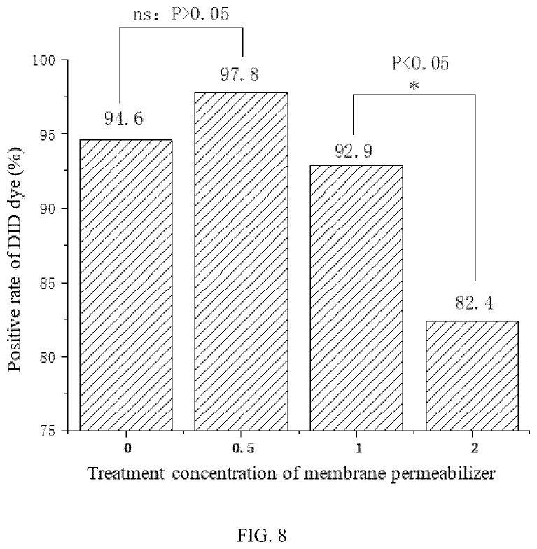

The comparison result of the fluorescence signal particle populations with scattered light signals after treatment with different concentrations of the membrane permeabilizers is shown in FIG. 8.

Conclusion: In Example 4, increasing the concentration of the membrane permeabilizer to 1× and 2× significantly reduced the positive rate of the DID membrane dye compared with the membrane permeabilizer concentrations of 0 and 0.5×. This suggested that the membrane permeabilizer may cause a small amount of rupture in the lentivirus; as the concentration of the membrane permeabilizer increased, the percentage of the DID membrane dye labeling slightly decreased, further demonstrating that the use of an appropriate concentration of the membrane permeabilizer in Example 3 is a key step in obtaining accurate determination results for particles of the lentivirus vector comprising the capsid and the VSV-G protein.

### Example 5: Detection by Flow NanoAnalyzer after Labeling with Nucleic Acid Dye and gB Antibody Conjugated to Fluorescent Dye

### 1) Dual Optical Labeling of Herpes Simplex Virus Vector

The concentrated solution of the herpes simplex virus vector product was 10-fold diluted with the PBS buffer to obtain solution 1, and 50 μL of solution 1 was incubated with 20 μL of the AF647-Anti-Glycoprotein B of HSV (gB) antibody at 37° C. for 30 min. The mixture was centrifuged at 4° C. at 100000 g for 80 min, the supernatant was discarded, and the pellet was resuspended in the PBS buffer. The mixture was centrifuged at 4° C. at 100000 g for 20 min, the supernatant was discarded, and 100 μL of 3 μM solution of SYTO9 in PBS buffer was added to resuspend the pellet to obtain the dual-labeled herpes simplex virus vector.

### 2) Detection of Dual-Labeled Herpes Simplex Virus Vector by Flow NanoAnalyzer

Flow NanoAnalyzer detection parameters were as follows: a laser detector 488 nm+638 nm; single-laser channel detection laser: 10/50 mW, 488; scattered light attenuation: 10%; detection pressure: 1 kpa; and signal type: under the condition of a small signal, and the FITC and PC5 dual fluorescence channel was used.

Detection of the dual-labeled herpes simplex virus vector: The dual-labeled herpes simplex virus vector obtained in step 1) of Example 5 was detected by the Flow NanoAnalyzer. The PC5 fluorescence was a nucleic acid dye excitation signal used for representing the g envelope glycoprotein in the lenti-herpes simplex virus vector, and the FITC fluorescence represented the nucleic acid of the herpes simplex virus vector in the lenti-herpes simplex virus vector. The FITC fluorescence channel detection results and the PC5 fluorescence channel detection results were analyzed, and the dot plot of the detection results was recorded. The resulting dot plot was gated into four quadrants, wherein
particles in a population with nucleic acid signals and gB signals were herpes simplex virus vectors comprising nucleic acids and envelopes;
particles in a population with gB signals but no nucleic acid signals were the empty capsids of the herpes simplex virus vectors;
particles in a population with nucleic acid signals but no gB signals were herpes simplex virus vectors comprising nucleic acids but lacking envelopes.

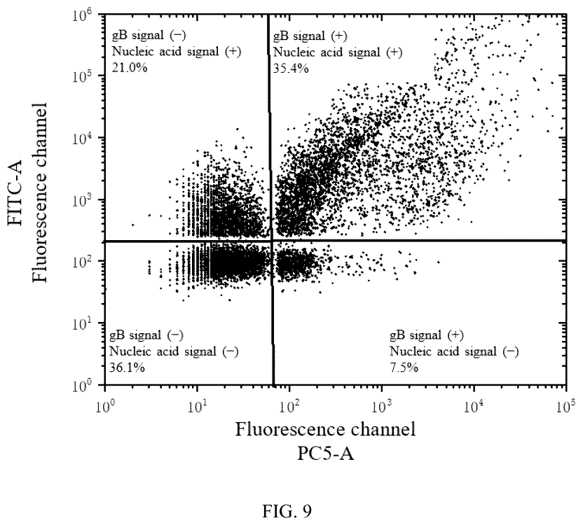

The results of the fluorescence signal particle populations are shown in FIG. 9.

Conclusion: The dual-labeled herpes simplex virus vector was detected by the Flow NanoAnalyzer, and it was found that the percentage of particles of the herpes simplex virus vector comprising the nucleic acid and the envelope in the herpes simplex virus vector product was 35.4%.

### Comparative Example 5: Detection by Flow NanoAnalyzer after Labeling with Antibodies Conjugated to Fluorescent Dye from Different Sources

### 1) Optical Labeling of Herpes Simplex Virus Vector

The concentrated solution of the herpes simplex virus vector product (HSV-1 or HSV-2) was 10-fold diluted with the PBS buffer to obtain solution 1, and 50 μL of solution 1 was separately incubated with 20 μL of antibodies conjugated to fluorescent dyes from different sources (AF647-Anti-HSV1+HSV2 gB antibody [10B7] (Anti-HSV1+HSV2 gB antibody [10B7] conjugated to the AF647 fluorescent dye, the manufacturer of the Anti-HSV1+HSV2 gB antibody [10B7]: Abcam), AF647-Herpes Simplex Virus Type 1/2 gB antibody (Herpes Simplex Virus Type 1/2 gB antibody conjugated to the AF647 fluorescent dye, the manufacturer of the Herpes Simplex Virus Type 1/2 gB antibody: Thermo Fisher), AF647-Anti-Glycoprotein B of HSV (gB) (herpes simplex virus envelope glycoprotein gB conjugated to the AF647 fluorescent dye, the manufacturer of the antibody: NanoFCM), and AF647-Anti-HSV1+HSV2 gD antibody (Anti-HSV1+HSV2 gD antibody conjugated to the AF647 fluorescent dye, the manufacturer of the Anti-HSV1+HSV2 gD antibody: Abcam)) at 37° C. for 30 min. The mixture was centrifuged at 4° C. at 100000 g for 80 min, the supernatant was discarded, and the pellet was resuspended in the PBS buffer. The mixture was centrifuged at 4° C. at 100000 g for 20 min, the supernatant was discarded, and 100 μL of the PBS buffer was added to resuspend the pellet to obtain the dual-labeled herpes simplex virus vector.

The preparation method for the above Anti-HSV1+HSV2 gB antibody conjugated to the AF647 fluorescent dye included the step of conjugating the AF647 fluorescent dye to the Anti-HSV1+HSV2 gB antibody according to the method in the instructions of the EZ-Link™ maleimide protein labeling kit to obtain the Anti-HSV1+HSV2 gB antibody conjugated to the AF647 fluorescent dye.

The preparation method for the above Herpes Simplex Virus Type 1/2 gB antibody conjugated to the AF647 fluorescent dye included the step of conjugating the AF647 fluorescent dye to the Herpes Simplex Virus Type 1/2 gB antibody according to the method in the instructions of the EZ-Link™ maleimide protein labeling kit to obtain the Herpes Simplex Virus Type 1/2 gB antibody conjugated to the AF647 fluorescent dye.

The manufacturer of the above Anti-HSV1+HSV2 gD antibody conjugated to the AF647 fluorescent dye was NanoFCM.

The preparation method for the above Anti-HSV1+HSV2 gD antibody conjugated to the AF647 fluorescent dye included the step of conjugating the AF647 fluorescent dye to the Anti-HSV1+HSV2 gD antibody according to the method in the instructions of the EZ-Link™ maleimide protein labeling kit to obtain the Anti-HSV1+HSV2 gD antibody conjugated to the AF647 fluorescent dye.

### 2) Detection of Herpes Simplex Virus Vector by Flow NanoAnalyzer

The dual-labeled herpes simplex virus vector obtained in step 1) of Comparative Example 5 was detected with the following detection parameters: Flow NanoAnalyzer detection parameters: a laser detector 488 nm+638 nm; single-laser channel detection laser: 10/50 mW, 488; scattered light attenuation: 10%; detection pressure: 1 kpa; and signal type: under the condition of a small signal, and the scattered channel and the FITC fluorescence channel/PC5 fluorescence channel were used. The dot plot of the detection results was recorded, and the resulting dot plot was gated into two quadrants, wherein
particles in a population with scattered light signals and fluorescent antibody expression signals were herpes simplex virus vectors with positive expression of the envelope-specific marker;
particles in a population with scattered light signals but no fluorescent antibody expression signals were herpes simplex virus vectors lacking expression of the envelope-specific marker.

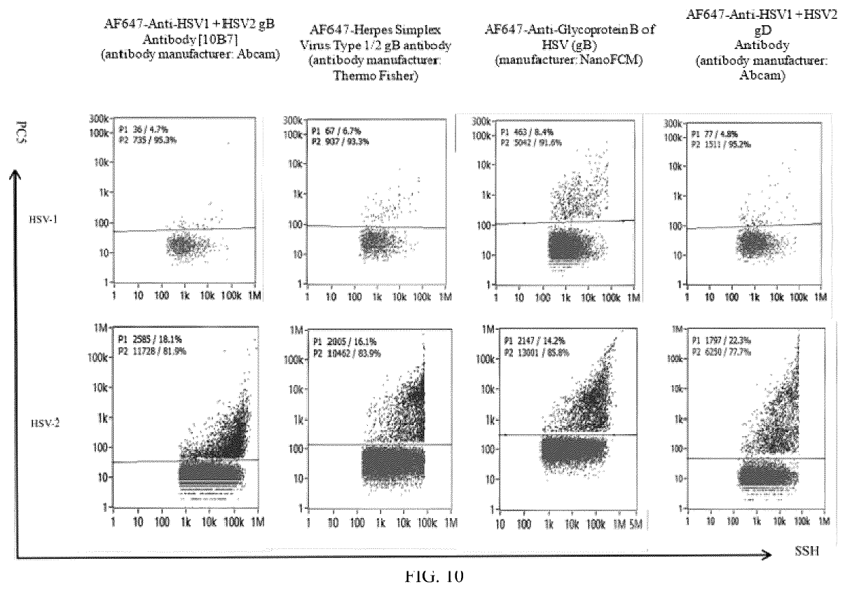

The comparison results of the percentages of particles of the herpes simplex virus vector with the positive expression of the envelope-specific marker were obtained, as shown in FIG. 10.

Conclusion: In Example 5 and Comparative Example 5, comparing the percentages of particles of the herpes simplex virus vector comprising envelope-specific marker expression obtained by labeling with AF647-Anti-HSV1+HSV2 gB antibody [10B7] (Anti-HSV1+HSV2 gB antibody [10B7] conjugated to the AF647 fluorescent dye, the manufacturer of the Anti-HSV1+HSV2 gB antibody [10B7]: Abcam), AF647-Herpes Simplex Virus Type 1/2 gB antibody (Herpes Simplex Virus Type 1/2 gB antibody conjugated to the AF647 fluorescent dye, the manufacturer of the Herpes Simplex Virus Type 1/2 gB antibody: Thermo Fisher), AF647-Anti-Glycoprotein B of HSV (gB) (herpes simplex virus envelope glycoprotein gB conjugated to the AF647 fluorescent dye, the manufacturer of the Anti-Glycoprotein B of HSV (gB): NanoFCM), and AF647-Anti-HSV1+HSV2 gD antibody (Anti-HSV1+HSV2 gD antibody conjugated to the AF647 fluorescent dye, the manufacturer of the Anti-HSV1+HSV2 gD antibody: Abcam)), it was found that the AF647-Anti-Glycoprotein B of HSV (gB) antibody in conjunction with SYTO9 provided the optimal results for simultaneous labeling of both the nucleic acid and the envelope of the herpes simplex virus vector.

### Example 6: Detection by Flow NanoAnalyzer after Labeling with Nucleic Acid Dye and Lipid Membrane Dye

### 1) Concentration Detection of Herpes Simplex Virus Vector

Flow NanoAnalyzer detection parameters were as follows: a laser detector 488 nm+638 nm; single-laser channel detection laser: 20/50 mW, 488 nm; scattered light attenuation: 0.2%; detection pressure: 1 kpa; and signal type: large, and the scattered channel was used.

Concentration detection of the herpes simplex virus vector product: The fluorescent microsphere standard with a concentration of 2.17×10¹⁰ particles/mL was 100-fold diluted to obtain a concentration standard solution. The concentrated solution of the herpes simplex virus vector product was 3-fold diluted to obtain a sample solution. The concentration standard solution and the sample solution were subjected to flow cytometry particle detection with the same detection parameters, and the number of particles was recorded at the same sample injection pressure and the same detection time as the detection of the concentration standard solution. Within the detection time, the number of particles in the sample solution was 3863 and the number of particles in the concentration standard solution was 7863. The concentration of the herpes simplex virus vector product was calculated based on the dilution factors, the concentration standard curve, and the detection results of the herpes simplex virus vector product, resulting in the concentration of 3.2×10⁸ particles/mL.

### 2) Dual Optical Labeling of Herpes Simplex Virus Vector

The concentrated solution of the herpes simplex virus vector product was 10-fold diluted with the buffer containing PBS (containing 136.89 mM NaCl, 2.67 mM KCl, 8.1 mM Na₂HPO₄, and 1.76 mM KH₂PO₄) and 0.5% Tween 20 to obtain solution 1, and 50 μL of solution 1 was incubated with 20 μL of the PKH67 lipid membrane dye (manufacturer: sigma) at 37° C. for 30 min. The mixture was centrifuged at 4° C. at 100000 g for 80 min, the supernatant was discarded, and the pellet was resuspended in the PBS buffer. The mixture was centrifuged at 4° C. at 100000 g for 20 min, the supernatant was discarded, and 100 μL of the PBS buffer containing 5 μM SYTO62 Red Nucleic Acid Stain was added to resuspend the pellet to obtain the dual-labeled herpes simplex virus vector.

### 3) Detection of Dual-Labeled Herpes Simplex Virus Vector by Flow NanoAnalyzer

Flow NanoAnalyzer detection parameters were as follows: a laser detector 488 nm+638 nm; single-laser channel detection laser: 10/50 mW, 488; scattered light attenuation: 10%; detection pressure: 1 kpa; and signal type: under the condition of a small signal, and the scattered channel and the FITC and PC5 dual fluorescence channel were used.

Detection of the dual-labeled herpes simplex virus vector: The dual-labeled herpes simplex virus vector was detected by the Flow NanoAnalyzer. The FITC fluorescence was a nucleic acid dye excitation signal used for representing PKH67 in the herpes simplex virus vector, and the PC5 fluorescence represented the nucleic acid dye excitation signal used for representing the nucleic acid of the herpes simplex virus vector. The FITC fluorescence channel detection results and the PC5 fluorescence channel detection results of the particle populations with scattered light signals were analyzed, and the dot plot of the detection results was recorded. The resulting dot plot was gated into four quadrants, wherein
particles in a population with scattered light signals, SYTO62 signals, and PKH67 signals were herpes simplex virus vectors comprising nucleic acids and envelopes;
particles in a population with scattered light signals and PKH67 signals, but no SYTO62 signals were the empty capsids of the herpes simplex virus vectors;
particles in a population with scattered light signals and SYTO62 signals, but no PKH67 signals were herpes simplex virus vectors comprising nucleic acids but lacking envelopes.

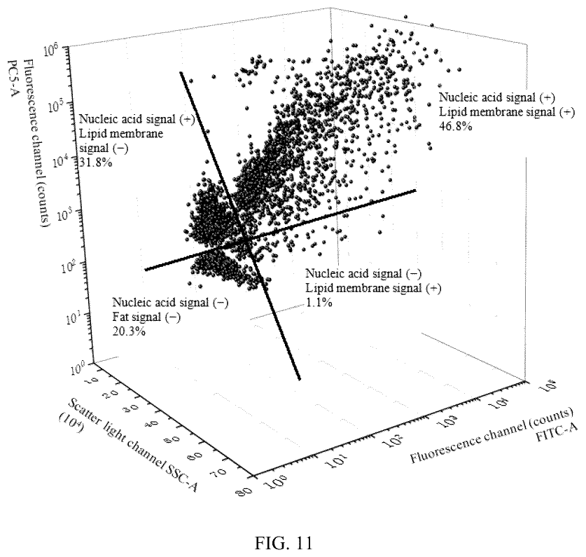

Result: The detection results from the scattered channel and the results of the fluorescence signal particle populations with scattered light signals are shown in FIG. 11.

Conclusion: The calculation of the bio-functional titer ratio of the herpes simplex virus vector product was completed, the particle population with a scattered light signal, a nucleic acid signal, and a lipid membrane signal was the herpes simplex virus vector comprising the nucleic acid and the lipid membrane, and the percentage of the particle population in the total number of particles with scattered light signals was 46.8%.

### Example 7: Detection by Flow NanoAnalyzer after Labeling with Nucleic Acid Dye and Spike Antibody Conjugated to Fluorescent Dye

1) Dual Optical Labeling of SARS-COV-2 (2019-nCOV) Spike Pseudovirus Vector

The concentrated solution of the SARS-COV-2 (2019-nCOV) Spike Pseudovirus vector product (1×10¹⁰ virus copies/mL) was 10-fold diluted with the PBS buffer to obtain solution 1, and 50 μL of solution 1 was incubated with 20 μL of the AF488-Anti-Spike protein [4A8], Human IgG1, Kappa antibody (an antibody of the Spike protein of SARS-COV-2 conjugated to AF488) at 37° C. for 30 min. The mixture was centrifuged at 4° C. at 100000 g for 80 min, the supernatant was discarded, and the pellet was resuspended in the PBS buffer. The mixture was centrifuged at 4° C. at 100000 g for 20 min, the supernatant was discarded, and 100 μL of the PBS buffer containing 5 μM SYTO62 Red Nucleic Acid Stain was added to resuspend the pellet to obtain the dual-labeled SARS-COV-2 (2019-nCOV) Spike Pseudovirus vector.

The preparation method for the above Anti-Spike protein [4A8], Human IgG1, Kappa antibody conjugated to the AF488 fluorescent dye included the step of conjugating the AF488 fluorescent dye to the Anti-Spike protein [4A8], Human IgG1, Kappa antibody according to the method in the instructions of the EZ-Link™ maleimide protein labeling kit to obtain the Anti-Spike protein [4A8], Human IgG1, Kappa antibody conjugated to the AF488 fluorescent dye.

2) Detection of Dual-Labeled SARS-COV-2 (2019-nCOV) Spike Pseudovirus Vector by Flow NanoAnalyzer

Flow NanoAnalyzer detection parameters were as follows: a laser detector 488 nm+638 nm; single-laser channel detection laser: 10/50 mW, 488; scattered light attenuation: 10%; detection pressure: 1 kpa; and signal type: under the condition of a small signal, and the scattered channel and the FITC and PC5 dual fluorescence channel were used.

Detection of the dual-labeled SARS-COV-2 (2019-nCOV) Spike Pseudovirus vector: The dual-labeled SARS-COV-2 (2019-nCOV) Spike Pseudovirus vector obtained in step 1) of Example 7 was detected by the Flow NanoAnalyzer. The FITC fluorescence was an AF488-Anti-Spike protein [4A8], Human IgG1, Kappa antibody excitation signal used for representing Spike in the SARS-COV-2 (2019-nCOV) Spike Pseudovirus vector, and the PC5 fluorescence represented a nucleic acid dye excitation signal used for representing the nucleic acid of the SARS-COV-2 (2019-nCOV) Spike Pseudovirus vector. The FITC fluorescence channel detection results and the PC5 fluorescence channel detection results of the particle populations with scattered light signals were analyzed, and the dot plot of the detection results was recorded. The resulting dot plot was gated into four quadrants, wherein
particles in a population with scattered light signals, SYTO62 signals, and Spike protein signals were SARS-COV-2 (2019-nCOV) Spike Pseudovirus vectors comprising nucleic acids and Spike proteins;
particles in a population with scattered light signals and Spike protein signals, but no SYTO62 signals were particles of the empty capsids of the SARS-COV-2 (2019-nCOV) Spike Pseudovirus vectors;
particles in a population with scattered light signals and SYTO62 signals, but no Spike protein signals were SARS-COV-2 (2019-nCOV) Spike Pseudovirus vectors comprising nucleic acids but lacking Spike proteins.

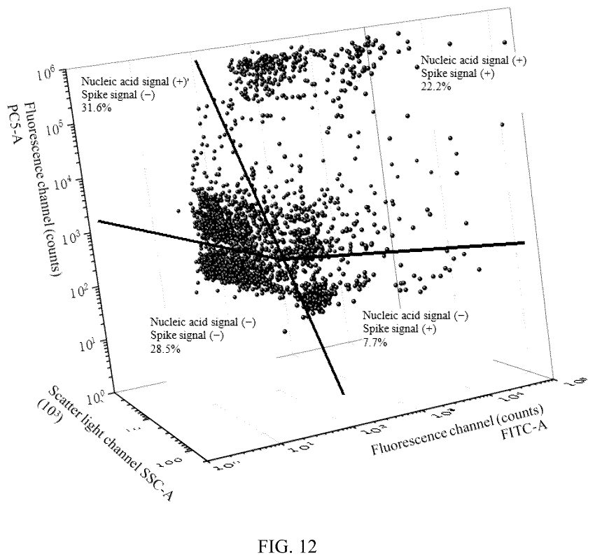

Result: The detection results from the scattered channel and the results of the fluorescence signal particle populations with scattered light signals are shown in FIG. 12.

Conclusion: The calculation of the bio-functional titer ratio of the SARS-COV-2 (2019-nCOV) Spike Pseudovirus was completed, and the particle population with a scattered light signal, a nucleic acid signal, and a Spike protein signal was the SARS-COV-2 (2019-nCOV) Spike Pseudovirus comprising the nucleic acid and the Spike protein, which accounted for 22.2% of the total number of particles with scattered light signals.

### Example 8: Detection by Flow NanoAnalyzer after Labeling with Nucleic Acid Dye and Gag Antibody Conjugated to Fluorescent Dye

### 1) Dual Optical Labeling of Retrovirus Vector

The concentrated solution of the retrovirus vector product was 10-fold diluted with a 10 mM PBS buffer to obtain solution 1, and 50 μL of solution 1 was incubated with 20 μL of the AF488 HIV-1 gag-pol antibody (an antibody of the gag protein of the retrovirus conjugated to AF488) at 37° C. for 30 min. The mixture was centrifuged at 4° C. at 100000 g for 80 min, the supernatant was discarded, and the pellet was resuspended in PBS. The mixture was centrifuged at 4° C. at 100000 g for 20 min, the supernatant was discarded, and 100 μL of the 10 mM PBS buffer containing 3 μM SYTO62 was added to resuspend the pellet to obtain the dual-labeled retrovirus vector.

The preparation method for the above HIV-1 gag-pol antibody conjugated to the AF488 fluorescent dye included the step of conjugating the AF488 fluorescent dye to the HIV-1 gag-pol antibody according to the method in the instructions of the EZ-Link™ maleimide protein labeling kit to obtain the HIV-1 gag-pol antibody conjugated to the AF488 fluorescent dye.

### 2) Detection of Dual-Labeled Retrovirus Vector by Flow NanoAnalyzer

Flow NanoAnalyzer detection conditions were as follows: a laser detector 488 nm+638 nm; single-laser channel detection laser: 10/50 mW, 488; scattered light attenuation: 10%; detection pressure: 1 kpa; and signal type: under the condition of a small signal, and the FITC and PC5 dual fluorescence channel was used.

Detection of the dual-labeled retrovirus vector: The dual-labeled retrovirus vector was detected by the Flow NanoAnalyzer. The PC5 fluorescence was a nucleic acid dye excitation signal used for representing the nucleic acid of the retrovirus vector, and the FITC fluorescence represented the gag protein in the retrovirus vector. The FITC fluorescence channel detection results and the PC5 fluorescence channel detection results were analyzed, and the dot plot of the detection results was recorded. The resulting dot plot was gated into four quadrants, wherein
particles in a population with nucleic acid signals and gag signals were retrovirus vectors comprising nucleic acids and capsids;
particles in a population with gag signals but no nucleic acid signals were the empty capsids of the retrovirus vectors;
particles in a population with nucleic acid signals but no gag signals were retrovirus vectors comprising nucleic acids but lacking capsids.

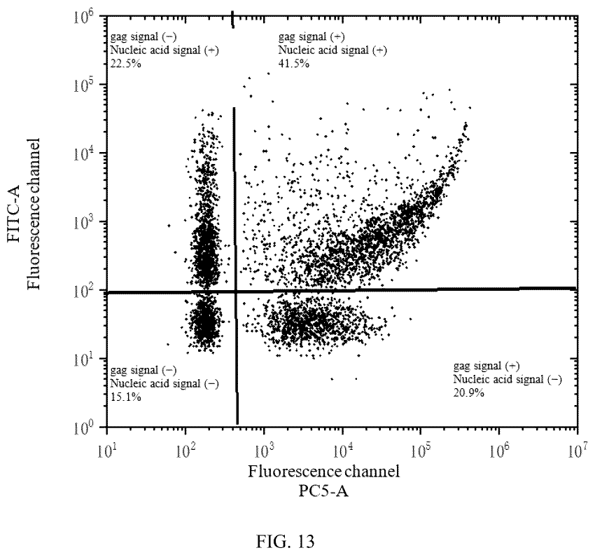

The results of the fluorescence signal particle populations are shown in FIG. 13.

Conclusion: The dual-labeled retrovirus vector was detected by the Flow NanoAnalyzer, and it was found that the percentage of particles of the retrovirus vector comprising the nucleic acid and the capsid in the retrovirus vector product was 41.5%.

### Example 9: Detection by Flow NanoAnalyzer after Labeling with Nucleic Acid Dye and Env Antibody Conjugated to Fluorescent Dye

### 1) Dual Optical Labeling of Retrovirus Vector

The concentrated solution of the retrovirus vector product was 10-fold diluted with a 10 mM PBS buffer to obtain solution 1, and 50 μL of solution 1 was incubated with 20 μL of the AF488 HIV-1 env Antibody antibody (an antibody of the env protein of the retrovirus conjugated to AF488) at 37° C. for 30 min. The mixture was centrifuged at 4° C. at 100000 g for 80 min, the supernatant was discarded, and the pellet was resuspended in PBS. The mixture was centrifuged at 4° C. at 100000 g for 20 min, the supernatant was discarded, and 100 μL of the 10 mM PBS buffer containing 3 μM SYTO62 was added to resuspend the pellet to obtain the dual-labeled retrovirus vector.

The preparation method for the above HIV-1 env Antibody antibody conjugated to the AF488 fluorescent dye included the step of conjugating the AF488 fluorescent dye to the HIV-1 env Antibody antibody according to the method in the instructions of the EZ-Link™ maleimide protein labeling kit to obtain the HIV-1 env Antibody antibody conjugated to the AF488 fluorescent dye.

### 2) Detection of Dual-Labeled Retrovirus Vector by Flow NanoAnalyzer

Flow NanoAnalyzer detection conditions were as follows: a laser detector 488 nm+638 nm; single-laser channel detection laser: 10/50 mW, 488; scattered light attenuation: 10%; detection pressure: 1 kpa; and signal type: under the condition of a small signal, and the FITC and PC5 dual fluorescence channel was used.

Detection of the dual-labeled retrovirus vector: The dual-labeled retrovirus vector was detected by the Flow NanoAnalyzer. The PC5 fluorescence was a nucleic acid dye excitation signal used for representing the nucleic acid of the retrovirus vector, and the FITC fluorescence represented the env protein in the retrovirus vector. The FITC fluorescence channel detection results and the PC5 fluorescence channel detection results were analyzed, and the dot plot of the detection results was recorded. The resulting dot plot was gated into four quadrants, wherein
particles in a population with nucleic acid signals and env signals were particles of the retrovirus vector comprising a nucleic acid and an envelope;
particles in a population with env signals but no nucleic acid signals were the empty capsids of the retrovirus vectors;
particles in a population with nucleic acid signals but no env signals were retrovirus vectors comprising nucleic acids but lacking envelopes.

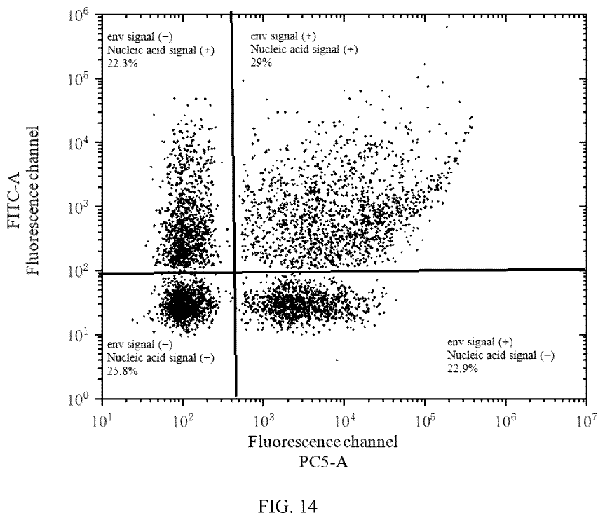

The results of the fluorescence signal particle populations are shown in FIG. 14.

Conclusion: The dual-labeled retrovirus vector was detected by the Flow NanoAnalyzer, and it was found that the percentage of particles of the retrovirus vector comprising the nucleic acid and the envelope in the retrovirus vector product was 29%.

The method of the present disclosure has been described by preferred examples. It will be apparent to those skilled in the art that the method and application described herein can be implemented and applied with modification or with appropriate modification and combination within the content, spirit, and scope of the present disclosure. Those skilled in the art can modify the process parameters appropriately in view of the disclosure herein. It is specifically noted that all such substitutions and modifications will be apparent to those skilled in the art and are intended to be included herein.

What is claimed is:

1. A method for analyzing one or more parameters of a viral nucleic acid vector, comprising:
step (1): preparing a fluorescence labeling reagent that can specifically bind to the viral nucleic acid vector and mixing the fluorescence labeling reagent with a sample comprising the viral nucleic acid vector;
the fluorescence labeling reagent comprising a nucleic acid probe, a nucleic acid stain, and at least one selected from a lipid membrane dye and a fluorophore-conjugated target recognition reagent; and the parameter comprising a viral capsid protein marker signal, and/or a viral envelope marker signal, as well as a nucleic acid signal or a target nucleic acid signal; preferably, the parameter comprising a nucleic acid signal or a target nucleic acid signal and an envelope protein marker signal;
step (2): detecting the sample solution comprising the viral nucleic acid vector by a flow particle analyzer, and recording data in a scattered channel and/or fluorescence channel; and
step (3): analyzing the data and calculating results of the parameters,
wherein the viral nucleic acid vector comprises a lentivirus vector, a retrovirus vector, a herpes simplex virus vector, and a recombinant coronavirus vector.

2. The method according to claim 1, wherein the parameter comprises a bio-functional titer of the viral nucleic acid vector; the bio-functional titer of the viral nucleic acid vector comprising: a titer of a viral nucleic acid vector comprising a nucleic acid, a viral capsid, and an envelope, a titer of a viral nucleic acid vector comprising a nucleic acid and an envelope, a titer of a viral nucleic acid vector comprising a nucleic acid and a viral capsid, a titer of a viral nucleic acid vector comprising a target nucleic acid, a viral capsid, and an envelope, a titer of a viral nucleic acid vector comprising a target nucleic acid and an envelope, a titer of a viral nucleic acid vector comprising a target nucleic acid and a viral capsid, and a titer of an empty capsid of a viral nucleic acid vector.

3. The method according to claim 1, wherein the parameter comprises a bio-functional titer of the lentivirus vector, the bio-functional titer of the lentivirus vector comprising: a titer of a lentivirus vector comprising a nucleic acid, a lentivirus capsid, and an envelope, a titer of a lentivirus vector comprising a target nucleic acid, a lentivirus capsid, and an envelope, a titer of a lentivirus vector comprising a nucleic acid and a VSV-G protein, a titer of a lentivirus vector comprising a target nucleic acid and a VSV-G protein, a titer of a lentivirus vector comprising a nucleic acid and an envelope, a titer of a lentivirus vector comprising a target nucleic acid and an envelope, a titer of an empty capsid of a lentivirus vector, a titer of a lentivirus vector comprising a nucleic acid but lacking a VSV-G protein, a titer of a lentivirus vector comprising a target nucleic acid but lacking a VSV-G protein, a titer of a lentivirus vector comprising a nucleic acid but lacking an envelope, a titer of a lentivirus vector comprising a target nucleic acid but lacking an envelope, a titer of a lentivirus vector comprising a nucleic acid and a lentivirus capsid, a titer of a lentivirus vector comprising a target nucleic acid and a lentivirus capsid, a titer of a lentivirus vector comprising a nucleic acid but lacking a lentivirus capsid, a titer of a lentivirus vector comprising a target nucleic acid but lacking a lentivirus capsid, a titer of a lentivirus vector comprising a nucleic acid and a p24 protein, a titer of a lentivirus vector comprising a target nucleic acid and a p24 protein, a titer of a lentivirus vector comprising a nucleic acid but lacking a p24 protein, a titer of a lentivirus vector comprising a target nucleic acid but lacking a p24 protein, a titer of a lentivirus vector comprising a nucleic acid and an envelope but lacking a lentivirus capsid, a titer of a lentivirus vector comprising a target nucleic acid and an envelope but lacking a lentivirus capsid, a titer of a lentivirus vector comprising a nucleic acid and a lentivirus capsid but lacking an envelope, a titer of a lentivirus vector comprising a target nucleic acid and a lentivirus capsid but lacking an envelope, a titer of a lentivirus vector comprising a nucleic acid but lacking a lentivirus capsid and an envelope, or a titer of a lentivirus vector comprising a target nucleic acid but lacking a lentivirus capsid and an envelope.

4. The method according to claim 1, wherein the parameter comprises a bio-functional titer of the retrovirus vector, the bio-functional titer of the retrovirus vector comprising: a titer of a retrovirus vector comprising a nucleic acid, a gag capsid protein, and an env envelope protein, a titer of a retrovirus vector comprising a target nucleic acid, a gag capsid protein, and an env envelope protein, a titer of a retrovirus vector comprising a nucleic acid and an env envelope protein, a titer of a retrovirus vector comprising a target nucleic acid and an env envelope protein, a titer of an empty capsid of a retrovirus vector, a titer of a retrovirus vector comprising a nucleic acid but lacking an env envelope protein, a titer of a retrovirus vector comprising a target nucleic acid but lacking an env envelope protein, a titer of a retrovirus vector comprising a nucleic acid and a gag capsid protein, a titer of a retrovirus vector comprising a target nucleic acid and a gag capsid protein, a titer of a retrovirus vector comprising a nucleic acid but lacking a gag capsid protein, and a titer of a retrovirus vector comprising a target nucleic acid but lacking a gag capsid protein.

5. The method according to claim 1, wherein the parameter comprises a bio-functional titer of the herpes simplex virus vector, the bio-functional titer of the herpes simplex virus vector comprising: a titer of a herpes simplex virus vector comprising a nucleic acid and g envelope glycoprotein, a titer of a herpes simplex virus vector comprising a target nucleic acid and g envelope glycoprotein, a titer of an empty capsid of a herpes simplex virus vector, a titer of a herpes simplex virus vector comprising a nucleic acid but lacking g envelope glycoprotein, and a titer of a herpes simplex virus vector comprising a target nucleic acids but lacking g envelope glycoprotein.

6. The method according to claim 5, wherein the g envelope glycoprotein comprises any one of envelope glycoprotein gB, envelope glycoprotein gC, envelope glycoprotein gD, envelope glycoprotein gE, envelope glycoprotein gG, envelope glycoprotein gH, envelope glycoprotein gI, envelope glycoprotein gJ, envelope glycoprotein gL, envelope glycoprotein gM, and envelope glycoprotein gN, or a combination thereof.

7. The method according to claim 1, wherein the parameter comprises a bio-functional titer of the recombinant coronavirus vector, the bio-functional titer of the recombinant coronavirus vector comprising: a titer of a recombinant coronavirus vector comprising a nucleic acid and a SPIKE protein, a titer of a recombinant coronavirus vector comprising a target nucleic acid and a SPIKE protein, a titer of an empty capsid of a recombinant coronavirus vector, a titer of a recombinant coronavirus vector comprising a nucleic acid but lacking a SPIKE protein, and a titer of a recombinant coronavirus vector comprising a target nucleic acid but lacking a SPIKE protein.

8. The method according to claim 1, further comprising the following steps:
step (a): before step (2), preparing a concentration standard solution, detecting the concentration standard solution by the flow particle analyzer, and recording scattering and/or fluorescent data of the concentration standard solution and/or a sample flow rate of the flow particle analyzer to measure the particle concentration of the sample comprising the viral nucleic acid vector; and/or
step (b): before step (2), preparing a particle size standard solution comprising particle size standards with different particle sizes, detecting the particle size standard solution by the flow particle analyzer, and recording scattering data of the particle size standards to measure the particle size of each particle and the particle size distribution of the viral nucleic acid vector.

9. The method according to claim 1, wherein the operation of mixing the fluorescence labeling reagent with the sample comprising the viral nucleic acid vector in step (1) further comprises adding a surfactant to mix with the sample comprising the viral nucleic acid vector;
optionally, the surfactant comprises at least one of an anionic surfactant, a nonionic surfactant, an amphoteric surfactant, and a cationic surfactant;
optionally, the surfactant comprises at least one from a Tween surfactant, Triton X-100, sodium dodecyl sulfate, nonylphenol polyoxyethylene ether, and a polyoxyethylene nonionic surfactant.

10. The method according to claim 8, wherein step (a) comprises: preparing the concentration standard solution by using beads of a known concentration; and/or
step (b) comprises: preparing the particle size standards by using beads with the same or similar refractive index to that of virus particles, calculating a median value of each particle size standard from images acquired by an electron microscope as an accurate value of each particle size standard, and preparing the particle size standard solutions comprising the particle size standards with different particle sizes.

11. The method according to claim 1, wherein the fluorophore-conjugated target recognition reagent comprises at least one from an antibody, an antibody fragment, an antibody analog, a lectin, an aptamer, a peptide, a growth factor, a glycolipid, a polysaccharide, a ligand, and a receptor.

12. The method according to claim 1, wherein the fluorophore-conjugated target recognition reagent comprises an antibody, an antibody fragment or an antibody analog, a ligand, and a receptor.

13. The method according to claim 11, wherein the antibody comprises at least one selected from a VSV-G antibody, a gB antibody, a gC antibody, a gD antibody, a gE antibody, a gG antibody, a gH antibody, a gI antibody, a gJ antibody, a gL antibody, a gM antibody, a gN antibody, an env antibody, a SPIKE antibody, an antibody specifically binding to a lentivirus capsid protein, an antibody specifically binding to a retrovirus capsid protein, an antibody specifically binding to a herpes simplex virus capsid protein, and an antibody specifically binding to a recombinant coronavirus capsid protein; and/or
the antibody specifically binding to the lentivirus capsid protein is selected from a p24 antibody; and/or
the antibody specifically binding to the retrovirus capsid protein is selected from a gag antibody; and/or
the nucleic acid probe comprises complementary sequences to the target nucleic acid or bases of the target nucleic acid, and a fluorescent dye is modified at the 5′ end or the 3′ end of the sequence or in the sequence; and/or
the nucleic acid stain comprises a cyanine dye, an impermeable dye, a permeable dye, an embedded dye, and a DNA double helix minor groove binding dye; and/or
the lipid membrane dye is selected from a lipophilic fluorescent dye with an affinity for a cell membrane and other lipid-soluble membrane structures; and/or
the fluorescent dye comprises a fluorescent molecule, a fluorescent material, or a combination thereof, and/or
the fluorescent dye comprises at least one of an organic fluorescent molecule, a fluorescent protein, a nucleic acid dye, a lipid membrane dye, quantum dots, and polymer dots; and/or
the lipid membrane dye is selected from DiD dye, DiO dye, Dil dye, DiR dye, DiA dye, Di-8-Anepps dye, Di-4-ANEPPS dye, PHK26 dye, PKH67 dye, CellMask Green dye, CellMask Orange dye, CellMask Red dye, CellVue Lavender dye, CellVue Plum dye, and CellVue NIR780 dye; and/or
the nucleic acid stain or the nucleic acid dye comprises at least one selected from Acridine Orange, Actinomycin D, 7-AAD (7-aminoactinomycin D), ACMA (9-amino-6-chloro-2-methoxyacridine), BOBO-1 Iodid, BOBO-3 Iodide, DAPI (4′,6-diamidino-2-phenylindole, dihydrochloride), dihydroethdium (hydrocthidine), Ethidium Homodimer-1 (EthD-1), Ethidium Homodimer-2 (EthD-2), Ethidium Monoazide Bromide (EMA), Hexidium Iodide, Hoechst 33258, Pentahydrate (bis-Benzimide), Hoechst 33342, Trihydrochloride, Trihydrate, Trihydrate-FluoroPure Grade, Hoechst 34580, LDS 751, NeuroTrace Blue Fluorescent Nissl Stain, NeuroTrace Green Fluorescent Nissl Stain, NeuroTrace 530/615 Red Fluorescent Nissl Stain, NeuroTrace Deep-Red Fluorescent Nissl Stain, POPO-1 Iodide, POPO-3 Iodide, PO-PRO-1 Iodide, propidium iodide, Propidium Iodide, OliGreen, PicoGreen, RiboGreen, SYBR Gold, SYBR Green I, SYBR Green II, SYBR Safe DNA gel stain, SYTO 40, SYTO 41, SYTO 42, SYTO 45, SYTO 9, SYTO 11, SYTO 12, SYTO 13, SYTO 14, SYTO 16, SYTO 21, SYTO 24, SYTO BC Green, SYTO 82, SYTO 83, SYTO 84, SYTO 85, SYTO 17, SYTO 59, SYTO 60, SYTO 61, SYTO 62, SYTO 63, SYTO 64, SYTO RNASelect, SYTOX Blue, SYTOX Green, SYTOX Orange, SYTOX Red, TO-PRO-1 Iodide, TO-PRO-3 Iodide, TOTO-1 Iodide, TOTO-3 Iodide, YO-PRO-1 Iodid, YO-PRO-3 Iodide, YOYO-1 Iodide, YOYO-3 Iodide, HCS NuclearMask Deep Red Stain, HCS NuclearMask Blue Stain, HCS NuclearMask Red Stain, and ethidium bromide.

14. The method according to claim 13, wherein the fluorophore-conjugated target recognition reagent is an antibody comprising at least one of a VSV-G antibody, an env envelope protein antibody, a g envelope glycoprotein antibody, a SPIKE protein antibody, a gag antibody, a p24 antibody, a viral capsid protein antibody, or an envelope protein antibody.

15. The method according to claim 13, wherein the fluorophore-conjugated target recognition reagent is a ligand and/or a receptor, and the ligand and/or receptor is at least one of a VSV-G ligand and/or receptor, an env envelope protein ligand and/or receptor, a g envelope glycoprotein ligand and/or receptor, a SPIKE protein ligand and/or receptor, a gag ligand and/or receptor, a p24 ligand and/or receptor, a viral capsid protein ligand and/or receptor, or an envelope protein ligand and/or receptor.

16. The method according to claim 13, the fluorescent dye comprises a fluorescent molecule, a fluorescent material, or a combination thereof, and the fluorescent dye is directly conjugated to the target recognition reagent; or the fluorescent dye is indirectly conjugated to the target recognition reagent with a recognition group comprising an antibody, an antigen, a receptor, or a polysaccharide, or any other particles or molecules, or any portions thereof, and the recognition group is capable of specifically recognizing the target recognition reagent.

17. The method according to claim 9, wherein mixing the fluorescence labeling reagent with the sample comprising the viral nucleic acid vector in step (1) comprises mixing the fluorescence labeling reagent and the surfactant with the sample comprising the viral nucleic acid vector; and/or
step (2) comprises photographing and counting the sample, and measuring a signal intensity value of each particle with a specific optical characteristic by the flow particle analyzer.

18. The method according to claim 1, wherein step (2) comprises using a flow particle analyzer with a scattered channel and at least two fluorescence channels or a flow particle analyzer with at least two fluorescence channels for detection, wherein one fluorescence channel is used for characterizing the nucleic acid or target nucleic acid signal, and the other fluorescence channel is used for characterizing the viral envelope marker signal; analyzing fluorescence signals of particles in a population with event-positive signals; recording and gating a dot plot, wherein
particles in a population with event signals, nucleic acid signals, and viral envelope marker signals are the viral nucleic acid vectors comprising the nucleic acids and the envelopes, or particles in a population with event signals, target nucleic acid signals, and viral envelope marker signals are the viral nucleic acid vectors comprising the target nucleic acids and the envelopes;
particles in a population with event signals and viral envelope marker signals, but no nucleic acid signals are the empty capsids of the viral nucleic acid vectors;
particles in a population with event signals and nucleic acid signals, but no viral envelope marker signals are viral nucleic acid vectors comprising nucleic acids but lacking envelopes, or particles in a population with event signals and target nucleic acid signals, but no viral envelope marker signals are the viral nucleic acid vectors comprising target nucleic acids but lacking envelopes.

19. The method according to claim 1, wherein step (2) comprises using a flow particle analyzer with a scattered channel and at least two fluorescence channels or a flow particle analyzer with at least two fluorescence channels, wherein one fluorescence channel is used for characterizing the nucleic acid signal or the target nucleic acid signal, and the other fluorescence channel is used for characterizing the viral capsid protein marker signal; analyzing the fluorescence signals of particles in a population with event signals; recording and gating a dot plot, wherein
particles in a population with event signals, nucleic acid signals, and viral capsid protein marker signals are the viral nucleic acid vectors comprising the nucleic acids and the lentivirus capsids, or particles in a population with event signals, target nucleic acid signals, and viral capsid protein marker signals are the viral nucleic acid vectors comprising the target nucleic acids and the viral capsids;
particles in a population with event signals and viral capsid protein marker signals, but no nucleic acid signals are the empty capsids of the viral nucleic acid vectors;
particles in a population with event signals and nucleic acid signals, but no viral capsid protein marker signals are viral nucleic acid vectors comprising nucleic acids but lacking viral capsids, particles in a population with event signals and target nucleic acid signals, but no viral capsid protein marker signals are viral nucleic acid vectors comprising target nucleic acids but lacking viral capsids.

20. The method according to claim 1, wherein step (2) comprises using a flow particle analyzer with a scattered channel and at least two fluorescence channels or a flow particle analyzer with at least one or two fluorescence channels, wherein at least one fluorescence channel is used for characterizing the nucleic acid signal or the target nucleic acid signal, and other channels are used for characterizing at least one viral capsid protein marker signal or at least one viral envelope marker signal; analyzing the fluorescence signals of particles in a population with event-positive signals; recording detection results in single or multiple dot plots; and gating the obtained dot plots, wherein
particles in a population with event signals, nucleic acid signals, viral capsid protein marker signals, and viral envelope marker signals are the viral nucleic acid vectors comprising the nucleic acids, the viral capsids, and the envelopes, or particles in a population with event signals, target nucleic acid signals, viral capsid protein marker signals, and viral envelope marker signals are the viral nucleic acid vectors comprising the target nucleic acids, the viral capsids, and the envelopes;
particles in a population with event signals but no nucleic acid signals are the empty capsids of the viral nucleic acid vectors, the particles in the population of the empty capsids of the viral nucleic acid vectors comprising at least one of the following particle populations: a) particles in a population of the empty capsids of viral nucleic acid vectors with event signals and viral envelope marker signals, but no nucleic acid signals and viral capsid protein marker signals; b) particles in a population of the empty capsids of viral nucleic acid vectors with event signals and viral capsid protein marker signals, but no nucleic acid signals and viral envelope marker signals; c) particles in a population of the empty capsids of viral nucleic acid vectors with event signals, viral capsid protein marker signals, and viral envelope marker signals, but no nucleic acid signals; and d) particles in a population of the empty capsids of viral nucleic acid vectors with event signals, but no nucleic acid signals, viral capsid protein marker signals, and viral envelope marker signals;
particles in a population with event signals, nucleic acid signals, and viral envelope marker signals, but no viral capsid protein marker signals are viral nucleic acid vectors comprising nucleic acids and envelopes but lacking viral capsids; or particles in a population with event signals, target nucleic acid signals, and viral envelope marker signals, but no viral capsid protein marker signals are viral nucleic acid vectors comprising target nucleic acids and envelopes but lacking viral capsids;
particles in a population with event signals, nucleic acid signals, and viral capsid protein marker signals, but no viral envelope marker signals are viral nucleic acid vectors comprising nucleic acids and capsids but lacking envelopes, or particles in a population with event signals, target nucleic acid signals, and viral capsid protein marker signals, but no viral envelope marker signals are viral nucleic acid vectors comprising target nucleic acids and viral capsids but lacking envelopes;
particles in a population with event signals and nucleic acid signals, but no viral capsid protein marker signals and viral envelope marker signals are viral nucleic acid vectors comprising nucleic acids but lacking viral capsids and envelopes, or particles in a population with event signals and target nucleic acid signals, but no viral capsid protein marker signals and viral envelope marker signals are viral nucleic acid vectors comprising target nucleic acids but lacking viral capsids and envelopes; and/or
the fluorescence channel is used for recording fluorescence signals with a specific wavelength range from the emission of a detected target when absorbing the light with a specific wavelength range.

21. The method according to claim 1, when the viral nucleic acid vector is the lentivirus vector, step (2) comprises using a flow particle analyzer with a scattered channel and at least two fluorescence channels or a flow particle analyzer with at least two fluorescence channels, wherein one fluorescence channel is used for characterizing the nucleic acid or target nucleic acid signal, and the other fluorescence channel is used for characterizing the viral envelope marker signal; analyzing the fluorescence signals of particles in a population with event-positive signals; recording and gating a dot plot, wherein
particles in a population with event signals, nucleic acid signals, and VSV-G signals are the lentivirus vectors comprising the nucleic acids and the VSV-G proteins, or particles in a population with event signals, target nucleic acid signals, and VSV-G signals are the lentivirus vectors comprising the target nucleic acids and the VSV-G proteins;
particles in a population with event signals and VSV-G signals, but no nucleic acid signals are the empty capsids of the lentivirus vectors;
particles in a population with event signals and nucleic acid signals, but no VSV-G signals are the lentivirus vectors comprising the nucleic acids but lacking the VSV-G proteins, or particles in a population with event signals and target nucleic acid signals, but no VSV-G signals are the lentivirus vectors comprising the target nucleic acids but lacking the VSV-G proteins;
and/or
when the viral nucleic acid vector is the retrovirus vector, step (2) comprises using a flow particle analyzer with a scattered channel and at least two fluorescence channels or a flow particle analyzer with at least two fluorescence channels, wherein one fluorescence channel is used for characterizing the nucleic acid or target nucleic acid signal, and the other fluorescence channel is used for characterizing the viral envelope marker signal; analyzing the fluorescence signals of particles in a population with event-positive signals; recording and gating a dot plot, wherein
particles in a population with event signals, nucleic acid signals, and env envelope protein signals are the retrovirus vectors comprising the nucleic acids and the env envelope proteins, or particles in a population with event signals, target nucleic acid signals, and env envelope protein signals are the retrovirus vectors comprising the target nucleic acids and the env envelope proteins;
particles in a population with event signals, env envelope protein signals, and nucleic acid signals are the empty capsids of the retrovirus vectors;
particles in a population with event signals and nucleic acid signals, but no env envelope protein signals are the retrovirus vectors comprising the nucleic acids but lacking the env envelope proteins, or particles in a population with event signals and target nucleic acid signals, but no env envelope protein signals are the retrovirus vectors comprising the target nucleic acids but lacking the env envelope proteins;
and/or
when the viral nucleic acid vector is the herpes simplex virus vector, step (2) comprises using a flow particle analyzer with a scattered channel and at least two fluorescence channels or a flow particle analyzer with at least two fluorescence channels, wherein one fluorescence channel is used for characterizing the nucleic acid or target nucleic acid signal, and the other fluorescence channel is used for characterizing the viral envelope marker signal; analyzing the fluorescence signals of particles in a population with event-positive signals; recording and gating a dot plot, wherein
particles in a population with event signals, nucleic acid signals, and g envelope glycoprotein signals are the herpes simplex virus vectors comprising the nucleic acids and the g envelope glycoproteins, or particles in a population with event signals, target nucleic acid signals, and g envelope glycoprotein signals are the herpes simplex virus vectors comprising the target nucleic acids and the g envelope glycoproteins;
particles in a population with event signals and g envelope glycoprotein signals, but no nucleic acid signals are the empty capsids of the herpes simplex virus vectors;
particles in a population with event signals and nucleic acid signals, but no g envelope glycoprotein signals are the herpes simplex virus vectors comprising the nucleic acids but lacking the g envelope glycoproteins, or particles in a population with event signals and target nucleic acid signals, but no g envelope glycoprotein signals are the herpes simplex virus vectors comprising the target nucleic acids but lacking the g envelope glycoproteins;
and/or
when the viral nucleic acid vector is the recombinant coronavirus vector, step (2) comprises using a flow particle analyzer with a scattered channel and at least two fluorescence channels or a flow particle analyzer with at least two fluorescence channels, wherein one fluorescence channel is used for characterizing the nucleic acid or target nucleic acid signal, and the other fluorescence channel is used for characterizing the viral envelope marker signal; analyzing the fluorescence signals of particles in a population with event-positive signals; recording and gating a dot plot, wherein
particles in a population with event signals, nucleic acid signals, and SPIKE protein signals are the recombinant coronavirus vectors comprising the nucleic acids and the SPIKE proteins, or particles in a population with event signals, target nucleic acid signals, and SPIKE protein signals are the recombinant coronavirus vectors comprising the target nucleic acids and the SPIKE proteins;
particles in a population with event signals and SPIKE protein signals, but no nucleic acid signals are the empty capsids of the recombinant coronavirus vectors;
particles in a population with event signals and nucleic acid signals, but no SPIKE protein signals are the recombinant coronavirus vectors comprising the nucleic acids but lacking the SPIKE proteins, or particles in a population with event signals and target nucleic acid signals, but no SPIKE protein signals are the recombinant coronavirus vectors comprising the target nucleic acids but lacking the SPIKE proteins;
when the viral nucleic acid vector is the lentivirus vector, step (2) comprises using a flow particle analyzer with a scattered channel and at least two fluorescence channels or a flow particle analyzer with at least two fluorescence channels, wherein preferably, one fluorescence channel is used for characterizing the nucleic acid signal or the target nucleic acid signal, and the other fluorescence channel is used for characterizing a lentivirus p24 protein signal when detecting the lentivirus vector; analyzing the fluorescence signals of particles in a population with event-positive signals; recording and gating a dot plot, wherein
particles in a population with event signals, nucleic acid signals, and lentivirus p24 protein signals are the lentivirus vectors comprising the nucleic acids and the lentivirus p24 proteins, or particles in a population with event signals, target nucleic acid signals, and lentivirus p24 protein signals are the lentivirus vectors comprising the target nucleic acids and the p24 proteins;
particles in a population with event signals and lentivirus p24 protein signals, but no nucleic acid signals are the empty capsids of the lentivirus vectors;
particles in a population with event signals and nucleic acid signals, but no lentivirus p24 protein signals are the lentivirus vectors comprising the nucleic acids but lacking the p24 proteins, or particles in a population with event signals and target nucleic acid signals, but no p24 protein signals are the lentivirus vectors comprising the target nucleic acids but lacking the p24 proteins;
and/or
when the viral nucleic acid vector is the retrovirus vector, step (2) comprises using a flow particle analyzer with a scattered channel and at least two fluorescence channels or a flow particle analyzer with at least two fluorescence channels, wherein preferably, one fluorescence channel is used for characterizing the nucleic acid signal or the target nucleic acid signal, and the other fluorescence channel is used for characterizing gag capsid protein signals when detecting the retrovirus vector; analyzing the fluorescence signals of particles in a population with event-positive signals; recording and gating a dot plot, wherein
particles in a population with event signals, nucleic acid signals, and gag capsid protein signals are the retrovirus vectors comprising the nucleic acids and the gag capsid proteins, or particles in a population with event signals, target nucleic acid signals, and gag capsid protein signals are the retrovirus vectors comprising the target nucleic acids and the gag capsid proteins;
particles in a population with event signals and gag capsid protein signals, but no nucleic acid signals are the empty capsids of the retrovirus vectors;
particles in a population with event signals and nucleic acid signals, but no gag capsid protein signals are the retrovirus vectors comprising the nucleic acids but lacking the gag capsid proteins, or particles in a population with event signals and target nucleic acid signals, but no gag capsid protein signals are the retrovirus vectors comprising the target nucleic acids but lacking the gag capsid proteins.

22. The method according to claim 8, wherein step (a) comprises detecting and recording the concentration standard solution and the sample solution comprising the viral nucleic acid vector at the same sample injection pressure in the same detection time by the flow particle analyzer, and
a calculation method for the particle concentration in the viral nucleic acid vector is as follows:
the particle concentration in the viral nucleic acid vector (particles/mL)=c×A1/A2,
wherein,
c is a concentration of the concentration standard solution, particles/mL;
A1 is the number of particles with event signals in the same unit time as A2 in the sample solution comprising the viral nucleic acid vector obtained by the detection gating from a scattered channel;
A2 is the number of particles with event signals in the same unit time as A1 in the concentration standard solution obtained by the detection gating from the scattered channel.

23. The method according to claim 1, wherein the calculation method for the particle concentration in the viral nucleic acid vector is as follows:
the particle concentration in the viral nucleic acid vector (particles/mL)=A1/K,
wherein,
K is a volumetric flow rate of the flow particle analyzer in a unit time;
A1 is the number of particles with event signals in the same unit time after conversion in the sample solution comprising the viral nucleic acid vector obtained by the detection gating from the scattered channel.

24. The method according to claim 10, wherein step (b) comprises detecting and recording the particle size standard solutions comprising the particle size standards with different particle sizes and the sample solution comprising the viral nucleic acid vector using the flow particle analyzer with the same detection settings and the same detection time, the detection settings comprising laser power and attenuation coefficient in a scattered channel; a calculation method for the particle size and the particle size distribution of the viral nucleic acid vector is to constructing a relation curve between average values of the scattered light intensity from the detection gating in the scattered channel and the particle size of the particle size standards, and then to calculate the particle size and the particle size distribution in the sample solution comprising the viral nucleic acid vector by using the scattered light intensity from the detection gating in the scattered channel of the viral nucleic acid vector in the sample solution and the constructed curve above.

25. The method according to claim 1, wherein a calculation method for a titer of a viral nucleic acid vector comprising a nucleic acid and an envelope protein is to multiply P1 and the particle concentration in the viral nucleic acid vector, wherein P1 is a percentage of the number of particles in a population with event signals, nucleic acid signals, and envelope protein signals in the total number of particles with event signals; and/or
a calculation method for the titer of the viral nucleic acid vector comprising the nucleic acid and the envelope is to multiply P1a and the particle concentration in the viral nucleic acid vector, wherein P1a is a percentage of the number of the particles in the population with the event signals, the nucleic acid signals, and the viral envelope marker signals in the total number of the particles with the event signals; and/or
a calculation method for the titer of the empty capsid of the lentivirus vector is to multiply P2 and the particle concentration in the viral nucleic acid vector, wherein P2 is a percentage of the number of particles in a population with event signals and envelope protein signals, but no nucleic acid signals in the total number of the particles with the event signals; or P2 is a percentage of the number of the particles in the population with the event signals and the viral capsid protein marker signals, but no nucleic acid signals in the total number of the particles with the event signals; or P2 is a percentage of the number of the particles in the population of the empty capsids of the lentivirus vectors with the event signals and the viral envelope marker signals, but no nucleic acid signals and viral capsid protein marker signals in the total number of the particles with the event signals; or P2 is a percentage of the number of the particles in the population of the empty capsids of the viral nucleic acid vectors with the event signals and the viral capsid protein marker signals, but no nucleic acid signals and viral envelope marker signals in the total number of the particles with the event signals; or P2 is a percentage of the number of the particles in the population of the empty capsids of the viral nucleic acid vectors with the event signals, the viral capsid protein marker signals, and the viral envelope marker signals, but no nucleic acid signals in the total number of the particles with the event signals; or P2 is a percentage of the number of the particles in the population of the empty capsids of the viral nucleic acid vectors with the event signals, but no nucleic acid signals, viral capsid protein marker signals, and viral envelope marker signals in the total number of the particles with the event signals; and/or
a calculation method for a titer of the viral nucleic acid vector comprising the nucleic acid but lacking the envelope is to multiply P3 and the particle concentration in the viral nucleic acid vector, wherein P3 is a percentage of the number of particles with event signals and nucleic acid signals, but no envelope signals in the total number of the particles with the event signals; and/or
a calculation method for the titer of the viral nucleic acid vector comprising the nucleic acid but lacking the envelope is to multiply P4 and the particle concentration in the viral nucleic acid vector, wherein P4 is a percentage of the number of the particles with the event signals and the nucleic acid signals, but no viral envelope marker signals in the total number of the particles with the event signals; and/or
a calculation method for a titer of the viral nucleic acid vector comprising the nucleic acid but lacking the viral capsid is to multiply P5 and the particle concentration in the viral nucleic acid vector, wherein P5 is a percentage of the number of the particles with the event signals and the nucleic acid signals, but no viral capsid protein marker signals in the total number of the particles with the event signals; and/or
a calculation method for a titer of a viral nucleic acid vector comprising a nucleic acid but lacking a capsid protein is to multiply P6 and the particle concentration in the viral nucleic acid vector, wherein P6 is a percentage of the number of particles with event signals and nucleic acid signals, but no viral capsid protein signals in the total number of the particles with the event signals; and/or
a calculation method for the titer of the lentivirus vector comprising the nucleic acid and the envelope but lacking the viral capsid is to multiply P7 and the particle concentration in the viral vector, wherein P7 is a percentage of the number of the particles in the population with the event signals, the nucleic acid signals, and the viral envelope signals, but no viral capsid protein signals in the total number of the particles with the event signals; and/or
a calculation method for a titer of the viral nucleic acid vector comprising the nucleic acid and the viral capsid but lacking the virus envelope is to multiply P8 and the particle concentration in the viral nucleic acid vector, wherein P8 is a percentage of the number of the particles in the population with the event signals, the nucleic acid signals, and the viral capsid protein signals, but no viral envelope signals in the total number of the particles with the event signals; and/or
a calculation method for a titer of the viral nucleic acid vector comprising the nucleic acid but lacking the viral capsid and the envelope is to multiply P9 and the particle concentration in the viral vector, wherein P9 is a percentage of the number of the particles in the population with the event signals and the nucleic acid signals, but no viral capsid protein signals and viral envelope signals in the total number of the particles with the event signals; and/or a calculation method for a titer of a viral nucleic acid vector comprising a target nucleic acid and an envelope protein is to multiply P1b and the particle concentration in the viral nucleic acid vector, wherein P1b is a percentage of the number of the particles in the population with the event signals, the target nucleic acid signals, and the envelope protein signals in the total number of the particles with the event signals; and/or
a calculation method for the titer of the viral nucleic acid vector comprising the target nucleic acid and the envelope is to multiply Plc and the particle concentration in the viral nucleic acid vector, wherein Plc is a percentage of the number of the particles in the population with the event signals, the target nucleic acid signals, and the viral envelope marker signals in the total number of the particles with the event signals; and/or
a calculation method for a titer of a viral nucleic acid vector comprising a target nucleic acid but lacking an envelope protein is to multiply P3a and the particle concentration in the viral nucleic acid vector, wherein P3a is a percentage of the number of particles with event signals and target nucleic acid signals, but no envelope protein signals in the total number of the particles with the event signals; and/or
a calculation method for the titer of the viral nucleic acid vector comprising the target nucleic acid but lacking the envelope is to multiply P4a and the particle concentration in the viral nucleic acid vector, wherein P4a is a percentage of the number of the particles with the event signals and the target nucleic acid signals, but no viral envelope marker signals in the total number of the particles with the event signals; and/or
a calculation method for a titer of the viral nucleic acid vector comprising the target nucleic acid but lacking the viral capsid is to multiply P5a and the particle concentration in the viral nucleic acid vector, wherein P5a is a percentage of the number of the particles with the event signals and the target nucleic acid signals, but no viral capsid protein marker signals in the total number of the particles with the event signals; and/or
a calculation method for a titer of a viral nucleic acid vector comprising a target nucleic acid but lacking a viral capsid protein is to multiply P6a and the particle concentration in the viral nucleic acid vector, wherein P6a is a percentage of the number of particles with event signals and target nucleic acid signals, but no viral capsid protein signals in the total number of the particles with the event signals; and/or
a calculation method for a titer of a viral nucleic acid vector comprising a target nucleic acid and an envelope but lacking a viral capsid protein is to multiply P7a and the particle concentration in the viral nucleic acid vector, wherein P7a is a percentage of the number of particles in a population with event signals, target nucleic acid signals, and viral envelope signals, but no viral capsid protein signals in the total number of the particles with the event signals; and/or
a calculation method for a titer of the viral nucleic acid vector comprising the target nucleic acid and the viral capsid but lacking the viral envelope is to multiply P8a and the particle concentration in the viral nucleic acid vector, wherein P8a is a percentage of the number of the particles in the population with the event signals, the target nucleic acid signals, and the viral capsid protein signals, but no viral envelope signals in the total number of the particles with the event signals; and/or
a calculation method for a titer of the viral nucleic acid vector comprising the target nucleic acid but lacking the viral capsid and the envelope is to multiply P9a and the particle concentration in the viral nucleic acid vector, wherein P9a is a percentage of the number of the particles in the population with the event signals and the target nucleic acid signals, but no viral capsid protein signals and viral envelope signals in the total number of the particles with the event signals.

26. The method according to claim 1, wherein the flow particle analyzer is a particle analysis detection instrument capable of achieving directional flow of a sample flow; and/or
a directional fluid system consists of loading unit and a flow unit; and/or
the particle analysis detection instrument comprises an optical system and a particle detector; and/or
the particle detector consists of a photoelectric sensor and a signal conditioning circuit with a function of band-limited filtering of high-frequency noise.
